(19) United States
(12) Patent Application Publication
(10) Pub. No.: US 2007/0105094 A1
(43) Pub. Date: May 10, 2007

(54) WATER-SOLUBLE CATIONIC MAGNETIC FINE PARTICLES AND METHOD FOR SEPARATING OR DETECTING LIPID VESICLE USING THE SAME

(75) Inventors: Masato Fujita, Chiba (JP); Noriyuki Ohnishi, Chiba (JP)

Correspondence Address: GREENBLUM & BERNSTEIN, P.L.C., 1950 ROLAND CLARKE PLACE, RESTON, VA, 20191, US

(73) Assignee: CHISSO CORPORATION, Osaka (JP)

(21) Appl. No.: 11/551,365

(22) Filed: Oct. 20, 2006

(30) Foreign Application Priority Data
Oct. 20, 2005 (JP) 2005/306008

(51) Int. Cl. C12Q 1/70

(52) U.S. Cl. 435/5

(57) ABSTRACT

A phospholipid vesicle such as a virus is to be rapidly separated (concentrated, roughly purified) and good detection (diagnosis) results can be obtained with suppressing inhibition of virus-denature, PCR inhibition, latex-aggregation inhibition, and the like. Moreover, the above operations are to be automated. A phospholipid vesicle is separated using a water-soluble cationic magnetic fine particle by a composite formation through a covalent bond or physical adsorption of a substance having a cationic functional group, a substance having a hydroxyl group, and a substance having magnetism.

### BACKGROUND OF THE INVENTION

1. Field of the Invention

The present invention relates to water-soluble cationic magnetic fine particles and a method for separating or detecting a body having a phospholipid membrane (hereinafter referred to phospholipid vesicle) using the same.

2. Background Art

In a diagnosis of a blood virus, a latex aggregation method using antibody beads is known. In this method, a support such as latex magnetic beads to which a monoclonal antibody or a polyclonal antibody against a protein present on the surface layer of the blood virus is immobilized is mixed with a blood sample which is expected to contain the blood virus. When the blood virus is not present in the blood sample, the latex beads maintain the dispersed state but when the blood virus is present, a membrane protein of the blood virus and the above antibody adsorbs each other to form an aggregate of the blood virus with the latex beads, so that the presence of the virus can be visually confirmed.

However, it is known that a blood sample contains various components such as proteins, polysaccharides, and low-molecular-weight compounds and ratios of the components may vary in every sample. A case is known where a result of detection exhibits pseudo positive or pseudo negative when a certain component is rich in the blood sample. In this case, a diagnostic system is constructed so that the case is usually designed not to be pseudo negative, but pseudo positive. In the diagnosis for HIV or the like, the latex aggregation method or a chromatographic method is first employed, which is convenient in operation of a blood virus or antibody content and enables rapid processing of many samples. However, a pseudo positive ratio in these diagnostic methods is about 0.3 and thus when judged as positive in the above diagnosis, it is necessary to confirm that the case is not pseudo positive through further diagnosis by other method. Moreover, immediately after virus infection, there is a problem that a term during which the content of the blood virus or the content of an antibody against the virus is very low and thus they cannot be detected (this term is called a window period) is long Therefore, when the above diagnosis is conducted within a short period from the time when the person being tested was infected, it is necessary to carrying out re-investigation after a certain period during which the blood virus or antibody content increases.

As a method of shortening the window period, a method of utilizing a polymerase chain reaction (PCR) has been developed. In this method, a highly sensitive detection is effected by amplifying a fragment specific to the virus among nucleic acids derived from the virus about 2 to 32 times, quantitatively determining the amount of the DNA fragment, and calculating back the virus content used in the PCR. When the nucleic acid derived from the virus is RNA, the detection of the virus can be achieved by carrying out a reverse transcription reaction to synthesize a DNA complementary to the RNA and subsequently carrying out PCR. These techniques are well known for those skilled in the art.

However, the diagnosis by PCR is very highly sensitive as compared with the above latex aggregation method or the like but there arises a problem that a certain component(s) in blood may inhibit PCR, so that there exist problems that pretreatment of the blood sample becomes complex and laborious and the processing time is prolonged.

As a procedure for solving such problems, a method for amplifying a nucleic acid without particular pretreatment has been developed, which includes addition of a reagent for neutralizing PCR-inhibitor(s) present in a sample However, since a quantitative result cannot be obtained when the amount of the PCR-inhibitor(s) in the sample is excessive to that of the neutralizing reagent, the method of treating the sample with the neutralizing reagent is applied only after the amount of the PCR-inhibitor(s) is reduced to some extent by conducting an operation such as aqueous two-phase separation.

In addition, as an inexpensive rough purification method of a virus for removing the inhibitor (s), there is known a method of using an anion exchange resin (e.g., cr. Patent Document 1). According to the method, a roughly purified virus is obtained by conducting gel filtration after cell lysis and centrifugation of hepatitis A virus substances from cultivation, passing the resultant eluate through an anion exchange resin composed of diethylaminoetyl-substituted cellulose to adsorb the virus and remove the inhibitor s), and then eluting the virus.

On the other hand, a virus detection method using magnetic beads is known (e.g., cf. Patent Document 2). In the method, a procedure is adopted wherein a virus-denatured solution is treated with cationic magnetic fine particles and a virus nucleic acid is directly absorbed on the magnetic beads to thereby remove the detection-inhibitor(s) and then the nucleic acid is released.

[Patent Document 1] JP-A-7-177882

[Patent Document 2] JP-T-2004-523238

However, in the case of the method of adding a reagent for neutralizing a PCR-inhibitor present in the above sample, there may be present an influence of the PCR-inhibitor and a case where a substance inhibiting denature of the virus by heating may remain in an amount more than that of the neutralizing agent, so that there is a problem that a diagnostic result of pseudo negative may be provided. Moreover, the method using an anion exchange resin described in the above Patent Document 1 is inexpensive but has problems that the operation is complex and laborious and the method is not suitable for processing many samples.

In addition to the viruses, infectious bacteria having an adverse effect on the human body have been known, and tuberculosis and sexually transmitted diseases may be exemplified as symptoms. The infection with these bacteria occurs from mucous membranes or wounded parts and they proliferate in the living body. In the case that these bacteria are to be target for test, diagnosis is conducted using blood or excrement such as phlegm or urine passing through an infected area or a washing liquid or a wiped matter of the infected area as a sample. Moreover, since antibodies against these bacteria are also produced in the living body, there is a method for diagnosing infection with the bacteria indirectly by measuring the amount of the antibodies in the blood. In these diagnoses, however, as in the case of the above concentration of the virus, inhibition of the detection by various components in the sample occurs, so that it is important to subject the sample to pretreatment at the diagnoses. When the pretreatment is not adequate, there is a possibility of a diagnostic result of pseudo negative.

As another problem, there is a case where ultracentrifugation operation is necessary as pretreatment for the above diagnosis but this step is extremely difficult to automate. As a means for solving the problem of the ultracentrifugation operation in automation, there is a method using magnetic beads.

On the other hand, in the virus detection method using magnetic beads described in the above Patent Document 2, the particle size of the magnetic fine particles to be used is so large as about 1 μm in order to facilitate magnetic separation and hence the fine particles may precipitate within several minutes, so that it is necessary to disperse the magnetic fine particles as homogeneous as possible by an operation such as stirring at automation. Thus, there is a problem that the mechanism of a device for automation is complicated. Furthermore, the magnetic fine particles are directly combined with a nucleic acid and hence it is anticipated that the nucleic acid is irreversibly adsorbed to some extent. In addition, since the nucleic acid is left in a free state for a long period of time, there are problems of possible contamination and decomposition.

### SUMMARY OF THE INVENTION

The invention solves any of the above problems associated with the conventional technologies. Particularly, in the separation or detection of a phospholipid vesicle membrane such as a virus or a bacterium, the invention provides a novel substance capable of reducing any detection-inhibiting causative substances by convenient and short-term processing and a method for separation or detection using the same.

As a result of extensive studies, the present inventors have found that the above problems are effectively solved by mixing water-soluble cationic magnetic fine particles, into which a cationic functional group capable of trapping a phospholipid vesicle under a homogeneous condition, with a liquid containing the phospholipid vesicle to form a phospholipid vesicle-cationic magnetic fine particle combined body which homogeneously disperses in the sample and further adding an aggregating agent capable of forming a molecular complex when mixed with the water-soluble cationic magnetic fine particles to form a water-insoluble composite, resulting in an aqueous two-phase partitioning. In the above steps, it is preferable to include a step of treating the sample containing the above water-soluble cationic magnetic fine particle-phospholipid vesicle combined body with a masking agent to form a combined body (masked body) of virus-cationic magnetic fine particle composite-masking agent which homogeneously disperses in the sample.

Namely, in the invention, a virus can be easily separated by conducting a step of adding an aggregating agent to an aqueous combined body (or masked body) containing the above phospholipid vesicle-cationic magnetic fine particles to convert the combined body (or masked body) into an aggregate (water-insoluble composite), a step of collecting the aggregate by a magnetic-separation operation to form pellets (aggregate pellets) and removing a supernatant containing inhibitor(s), and a step of re-dispersing the aggregate pellets into water or a buffer, sequentially. Thereby, the inhibitor(s) for virus diagnosis can be reduced and thus accuracy of the diagnosis can be enhanced.

Furthermore, in the invention, pretreatment for detecting a phospholipid vesicle can be automated

Namely, the invention comprises the following constitution.
[1] A water-soluble cationic magnetic fine particle comprising a substance having a cationic functional group, a substance having a hydroxyl group and a substance having magnetism, wherein the substance having a cationic functional group, the substance having a hydroxyl group and the substance having magnetism form a composite through a covalent bond or physical adsorption. The cationic magnetic fine particle of [1] preferably has a positive charge in an aqueous solution and preferably has an average particle size of 300 nm or less.
[2] The water-soluble cationic magnetic fine particle according to the above [1], wherein the substance having magnetism is at least one substance selected from the group consisting of metals, metal oxides and latex magnetic beads, the substance having a hydroxyl group is a substance having a polyol framework, and the substance having a cationic functional group is at least one functional group selected from the group consisting of primary amino groups, secondary amino groups, tertiary amino groups, quaternary ammonium groups and guanidino groups. In the cationic magnetic fine particles of [1], the substance having a polyol framework is preferably a polyol obtained by polymerization using a polysaccharide, a polysaccharide derivative, or a polymerizable monomer having a hydroxyl group as a composition.
[3] The water-soluble cationic magnetic fine particle according to the above [1] or [2], wherein the substance having magnetism is at least one substance selected from the group consisting of magnetite, maghemite, hematite, gesite and latex magnetic beads,

the substance having a hydroxyl group is at least one polyol selected from the group consisting of dextran, dextrin, cellulose, agarose, starch, carboxymethyl cellolose, hydroxyacetyl cellulose, diethylaminoethyl cellulose, pullulan, amylose, gellan, arabinose galactan, polyvinyl alcohol and polyallyl alcohol, or a polyol obtained by polymerizing at least one compound selected from the group consisting of vinyl alcohol, allyl alcohol, 2-hydroxyethyl(meth)acrylate, glycerol-mono(meth)acrylate, 4-hydroxybutyl acrylate, 3-hydroxybutyl acrylate, 3-hydroxypropyl acrylate, and 2-hydroxy-2-methylpropyl acrylate as a component of a polymerizable monomer composition, and

the substance having a cationic functional group is at least one substance selected from polyallylamine, polyvinylamine, polyethyleneimine, polylysine, polyguanidine, poly(N,N-dimethylaminoethyl(meth)acrylamide), poly(N,N-dimethylaminopropyl(meth)acrylamide), polyaminopropyl(meth)acrylamide, or a substance obtained by substituted with at least one compound selected from the group consisting of diethylaminoethyl chloride hydrochloride, ethylenediamine, hexamethylenediamine, tris(aminoethyl)amine, aziridine hydrochloride, aminopropyltriethoxysilane and aminoethylaminopropyltriethoxysilane.
[4] The water-soluble cationic magnetic fine particle according to any one of the above [1] to [3], wherein the substance having a cationic functional group is at least one substance selected from polyethyleneimine and polylysine, and the substance having a hydroxyl group is at least one substance selected from dextran and polyvinyl alcohol, and the substance having magnetism is at least one substance selected from magnetite and maghemite. Moreover, in the above cationic magnetic fine particles, there may be utilized cationic magnetic fine particles wherein the substance having a cationic functional group is immobilized into a structure where the substance having magnetism is coated with the substance having a hydroxyl group. Also, in the above cationic magnetic fine particles, there may be utilized cationic magnetic fine particles wherein the substance having a cationic functional group is immobilized into a structure where the substance having magnetism is coated with the substance having a hydroxyl group obtained by making an acidic aqueous solution containing a polyol and a metal ion alkaline. Furthermore, in the above cationic magnetic fine particles, there may be utilized water-soluble cationic magnetic fine particles obtained by introducing polyethyleneimine through reductive amination into dextran-coated magnetite having aldehyde obtained by treating, with sodium periodate, dextran-coated magnetite obtained by adding ammonia to an acidic aqueous solution containing dextran and iron chloride. Also, in the above cationic magnetic fine particles, there may be utilized water-soluble cationic magnetic fine particles obtained by reacting, with polylysine, polyvinyl alcohol-coated magnetite having a glycidyl group obtained by treating, with epichlorohydrin, polyvinyl alcohol-coated magnetite obtained by adding ammonia to an acidic aqueous solution containing polyvinyl alcohol and iron chloride.
[5] A combined body of a water-soluble cationic magnetic fine particle and a phospholipid vesicle, wherein the water-soluble cationic magnetic fine particle according to any one of the above [1] to [4] and a body having a phospholipid membrane (hereinafter referred to as phospholipid vesicle) are combined.
[6] The combined body according to the above [5], wherein the phospholipid vesicle is a virus, a bacterium, a fungus, or a true fungus. Furthermore, in the above [5], the following embodiments are preferable. Namely, in the above [5], the combined body wherein the phospholipid vesicle is influenza virus, cytemegalo virus, HIV, papilloma virus, respiratory syncytial virus, poliomyelitis virus, pox virus, measles virus, arbovirus, coxsackievirus, herpes virus, hantavirus, hepatitis virus, Lyme disease virus, mumps virus, and rotavirus. Moreover, in the above [5], the combined body wherein the phospholipid vesicle is a bacterium belonging to Genus Neisseria, Genus aerobcter, Genus Pseudomonas, Genus Porphyromonas, Genus Salmonella, Genus Escherichia, Genus Pasteurelle, Genus Shigella, Genus Bacillus, Genus Helicobacter, Genus Corynebacterium, Genus Clostridium, Genus Actinomycetes, Genus Yersinia, Genus Staphylococcus, Genus Vaudetera, Genus Brucella, Genus Vibrio, or Genus Streptococcus. Furthermore, in the above [5], the combined body wherein the phospholipid vesicle is hepatitis B virus. In addition, in the above [5], the combined body wherein the phospholipid vesicle is supplied as a component contained in at least one liquid selected from the group consisting of human body fluid, animal body fluid, a suspension of paranasal sinus-wiped matter, a suspension of local area-wiped matter, urine, saliva, phlegm, a suspension of feces, river water, and tap water. Also, in the above [5], the combined body which contains inside at least one selected from amino acids, oligopeptides, peptides, proteins, glycoproteins, lipoproteins, proteoglycans, monosaccharides, oligosaccharides, polysaccharides, lipopolysaccharides, fatty acids, eicosanoids, phospholipids, triglycerides, phospholipids, nucleosides, nucleotides, nucleic acids, DNA molecules, and RNA molecules.
[7] A combined body of a water-insoluble cationic magnetic fine particle, a phospholipid vesicle and a masking agent, wherein the combined body according to the above [5] or [6] and a masking agent are combined.
[8] The combined body according to the above [7], wherein the masking agent is a substance containing at least one acid structure selected from the group consisting of carboxylic acid, phosphoric acid, sulfuric acid, and boric acid. Moreover, in the above [7], there may be utilized a combined body wherein the masking agent is at least one masking agent selected from the group consisting of poly(meth)acrylic acid, polycarboxymethylstyrene, hyaluronic acid, α-polyglutamic acid, ω-polyglutamic acid, gelan, carboxymethyl cellulose, carboxymethyl dextran, polyphosphoric acid, poly(phosphoric acid sugar), nucleic acids, phosphoric acid, citric acid, polystyrylsulfuric acid, dextran sulfuric acid, and polystyrylboric acid. Furthermore, in the above [7], the masking agent is poly(meth)acrylic acid. In addition, in the above [7], there may be utilized a combined body wherein the masking agent is poly(meth)acrylic acid having an average molecular weight of 10,000 to 50,000.
[9] A composite of a water-insoluble cationic magnetic fine particle, a phospholipid vesicle and an aggregating agent, wherein the combined body according to the above [5] or [6] and an aggregating agent are combined.
[10] A composite of a water-insoluble cationic magnetic fine particle, a phospholipid vesicle, a masking agent and an aggregating agent, wherein the combined body according to the above [7] or [8] and an aggregating agent are combined.
[11] The composite according to the above [9] or [10], wherein the aggregating agent is a polyether.
[12] The composite according to the above [9] or [10], wherein the aggregating agent is at least one substance selected from the group consisting of a substance having a polyalkylene glycol structure in a main chain, a substance having a polyalkylene glycol structure in a side chain and a substance having a polyglycerin structure in a main chain. Moreover, in the above [9] or [10], there may be utilized a composite wherein the aggregating agent is polyethylene glycol. Furthermore, there may be utilized a composite wherein the aggregating agent is polyethylene glycol having an average molecular weight of 2,000 to 20,000. In addition, there may be utilized pellets of a composite wherein the water-insoluble cationic magnetic fine particle-phospholipid vesicle-aggregating agent composite is obtained by separating it from a liquid using at least one method selected from magnetic separation, centrifugation, and filtration. Also, there may be utilized an aqueous solution wherein pellets of the above-mentioned composite are re-dispersed.
[13] The composite according to the above [12], which is a composite of a cationic magnetic fine particle, a phospholipid vesicle, a masking agent and an aggregating agent,

wherein the cationic magnetic fine particle is a composite of dextran-coated magnetite and polyethyleneiminie,

the phospholipid vesicle is a virus,

the masking agent is at least one masking agent selected from the group consisting of poly(meth)acrylic acid, polycarboxymethylstyrene, hyaluronic acid, α-polyglutamic acid, ω-polyglutamic acid, gelan, carboxymethyl cellulose, carboxymethyl dextran, polyphosphoric acid, poly(phosphoric acid sugar), nucleic acids, phosphoric acid, citric acid, polystyrylsulfuric acid, dextran sulfuric acid and polystyrylboric acid, and

the aggregating agent is at least one aggregating agent selected from the group consisting of polyethylene glycol, polypropylene glycol, polyethyleneglycol-polypropylene glycol random copolymer, and polyethyleneglycol-polypropylene glycol block copolymer, polymethoxyethoxy(meth)acrylate, poly(diethylene glycol-(meth)acrylate-methyl ether), poly(triethylene glycol-(meth)acrylate-methyl ether), poly(tetraethylene glycol-(meth)acrylate-methyl ether), poly(polyethylene glycol (meth)acrylate), and random and block copolymers thereof, and poly(glycerin-2-ethyl ether), poly(glycerin-2-diethylene glycol methyl ether), poly(glycerin-2-triethylene glycol methyl ether), poly(glycerin-2-tetraethylene glycol methyl ether), poly(glycerin-2polyethylene glycol ether), poly(glycerin-2-polypropylene glycol ether), and poly(glycerin-2-polyethylene glycol ether) (glycerin-2-polypropylene glycol ether) copolymer.
[14] The composite according to the above [12], which is a composite of a cationic magnetic fine particle, a phospholipid vesicle, a masking agent and an aggregating agent,

wherein the cationic magnetic fine particle is a composite of magnetite coated with dextran having an average molecular weight of 3,000 to 100,000 and polyethyleneimine having an average molecular weight of 600 to 10,000,

the phospholipid vesicle is at least one virus selected from the group consisting of influenza virus, cytemegalo virus, HIV, papilloma virus, respiratory syncytial virus, poliomyelitis virus, pox virus, measles virus, arbovirus, coxsackievirus, herpes virus, hantavirus, hepatitis virus, Lyme disease virus, mumps virus and rotavirus,

the masking agent is poly(meth)acrylic acid having an average molecular weight of 10,000 to 50,000 or a salt thereof, and

the aggregating agent is polyethylene glycol having an average molecular weight of 2,000 to 20,000.

Moreover, in the above [12], there may be utilized a water-insoluble cationic magnetic fine particle-phospholipid vesicle-masking agent-aggregating agent composite, which is formed by mixing an water-soluble combined body of cationic magnetic fine particle-phospholipid vesicle-masking agent formed by mixing a water-soluble combined body of cationic magnetic fine particle-phospholipid vesicle obtained by mixing water-soluble cationic magnetic fine particles obtained by composite formation between magnetite coated with dextran having an average molecular weight of 10,000 to 40,000 and polyethyleneimine having an average molecular weight of 1,800 to 10,000 with a liquid containing at least one phospholipid vesicle selected from the group consisting of fungi, bacteria, and viruses, with an aqueous solution containing poly(meth)acrylic acid having an average molecular weight of 25,000 to 50,000, with polyethylene glycol having an average molecular weight of 5,000 to 10,000. Furthermore, in the above [12], there may be utilized a water-insoluble cationic magnetic fine particle-phospholipid vesicle-masking agent-aggregating agent composite, which is formed by mixing an water-soluble combined body of cationic magnetic fine particle-phospholipid vesicle-masking agent formed by mixing a water-soluble combined body of cationic magnetic fine particle-phospholipid vesicle obtained by mixing water-soluble cationic magnetic fine particles obtained by composite formation between magnetite coated with dextran having an average molecular weight of 40,000 and polyethyleneimine having an average molecular weight of 1,800 with a liquid containing at least one phospholipid-vesicle selected from the group consisting of fungi, bacteria, and viruses, with an aqueous solution containing poly(meth)acrylic acid having an average molecular weight of 25,000, with polyethylene glycol having an average molecular weight of 6,000 to 8,000. In addition, in the above [12], there may be utilized a water-insoluble cationic magnetic fine particle-phospholipid vesicle-masking agent-aggregating agent composite, which is formed by mixing an water-soluble combined body of cationic magnetic fine particle-phospholipid vesicle-masking agent formed by mixing a water-soluble combined body of cationic magnetic fine particle-phospholipid vesicle obtained by mixing water-soluble cationic magnetic fine particles obtained by composite formation between magnetite coated with dextran having an average molecular weight of 40,000 and polyethyleneimine having an average molecular weight of 1,800 with a liquid containing at least one phospholipid vesicle selected from the group consisting of fungi, bacteria, and viruses, with an aqueous solution containing poly(meth)acrylic acid having an average molecular weight of 25,000, with polyethylene glycol having an average molecular weight of 8,000. Also, in the above [12], there may be utilized a water-insoluble cationic magnetic fine particle-phospholipid vesicle-masking agent-aggregating agent composite, which is formed by mixing an water-soluble combined body of cationic magnetic fine particle-phospholipid vesicle-masking agent formed by mixing a water-soluble combined body of cationic magnetic fine particle-phospholipid vesicle obtained by mixing water-soluble cationic magnetic fine particles obtained by composite formation between magnetite coated with dextran having an average molecular weight of 40,000 and polyethyleneimine having an average molecular weight of 1,800 with a liquid containing hepatitis B virus, with an aqueous solution containing poly(meth)acrylic acid having an average molecular weight of 25,000, with polyethylene glycol having an average molecular weight of 8,000.
[15] A process for separating a phospholipid vesicle, comprising mixing an aqueous solution of a water-soluble cationic magnetic fine particle containing a substance having a cationic functional group, a substance having a hydroxyl group and a substance having magnetism, with a liquid containing a phospholipid vesicle, to form a water-soluble combined body of a cationic magnetic fine particle and a phospholipid vesicle.
[16] The process for separating a phospholipid vesicle according to the above [15], which further comprises mixing with a masking agent.
[17] The process for separating a phospholipid vesicle according to the above [15] or [16], which comprises:

an adsorption step of mixing a water-soluble cationic magnetic fine particle having a polyol and a substance having a cationic functional group in the structure, with a liquid containing a phospholipid vesicle to form a water-soluble combined body of a cationic magnetic fine particle and a phospholipid vesicle;

an aggregation step of mixing the water-soluble combined body with an aggregating agent to form a water-insoluble composite of a cationic magnetic fine particle, a phospholipid vesicle and an aggregating agent;

a separation step of forming a pellet of the water-insoluble composite by at least one method selected from magnetic separation, centrifugation and filtration and removing the resultant supernatant; and

a re-dispersion step of dispersing the pellet in a liquid.

Moreover, in the above [17], there may be utilized a process for separating a phospholipid vesicle wherein operations are conducted in the order of the steps.
[18] The process for separating a phospholipid vesicle according to the above [15] or [16], which comprises:

an adsorption step of mixing a water-soluble cationic magnetic fine particle having a polyol and a substance having a cationic functional group in the structure, with a liquid containing a phospholipid vesicle to form a water-soluble combined body of a cationic magnetic fine particle and a phospholipid vesicle;

a masking step of mixing the water-soluble combined body with an aqueous solution containing a masking agent to form a water-soluble combined body of a cationic magnetic fine particle, a phospholipid vesicle and a masking agent;

an aggregation step of mixing the water-soluble combined body of a cationic magnetic fine particle, a phospholipid vesicle and a masking agent, with an aggregating agent to form a water-insoluble composite of a cationic magnetic fine particle, a phospholipid vesicle, a masking agent and an aggregating agent;

a separation step of forming a pellet of the water-insoluble composite by at least one method selected from magnetic separation, centrifugation and filtration, and removing the resultant supernatant, and

a re-dispersion step of dispersing the pellet in a liquid.

Moreover, in the above [18], there may be utilized a process for separating a phospholipid vesicle wherein operations are conducted in the order of the steps.
[19] A process for detecting a virus, comprising a step of mixing a water-soluble cationic magnetic fine particle containing a substance having a cationic functional group, a substance having a hydroxyl group and a substance having magnetism, with a liquid containing a virus to form a water-soluble combined body of a cationic magnetic fine particle and a phospholipid vesicle.
[20] The process for detecting a virus according to the above [19], which comprises:

a step of mixing a water-soluble cationic magnetic particle, with a serum or plasma containing the virus to form a water-soluble combined body of a cationic magnetic fine particle and virus, in which the water-soluble cationic magnetic particle is obtained by treating a water-soluble dextran magnetite with a periodate to form a dextran magnetite having an aldehyde and then covalently bonding the dextran magnetite having an aldehyde through reductive amination with polyethyleneimine having an average molecular weight of 1,800 which is a substance having a cationic functional group;

a step of mixing the water-soluble combined body with an aqueous solution of polyacrylic acid having an average molecular weight of 25,000 to form a water-soluble combined body of a cationic magnetic fine particle, virus and polyacrylic acid;

a step of further mixing the water-soluble combined body of a cationic magnetic fine particle, virus and polyacrylic acid, with an aqueous solution of polyethylene glycol having a molecular weight of 6,000 to 8,000 to form a water-insoluble composite of a cationic magnetic fine particle, virus, polyacrylic acid and polyethylene glycol;

a step of forming a pellet of the water-insoluble composite by magnetic collection and removing the resultant supernatant;

a step of dispersing the pellet in a nucleic acid amplification reaction solution;

a step of denaturing the virus in the pellet by heating to release nucleic acids of the virus; and

a step of amplifying the virus nucleic acids by a nucleic acid amplification reaction (PCR, ICAN).

Moreover, in the above [19], there may be utilized a process for detecting a virus, which comprises a step of treating a water-soluble dextran magnetite with a periodic acid to form a dextran magnetite having an aldehyde and then covalently bonding it through reductive amination with polyethyleneimine having an average molecular weight of 600 to 70,000, which is a polycation, to form a water-soluble cationic magnetic fine particles; a step of mixing the cationic magnetic fine particles with a serum containing the virus to form a water-soluble combined body of cationic magnetic fine particle-virus; a step of mixing the combined body with an aqueous solution of polyacrylic acid having an average molecular weight of 5,000 to 100,000 to form a water-soluble combined body of cationic magnetic fine particle-virus-polyacrylic acid; a step of further mixing the combined body with an aqueous solution of polyethylene glycol having a molecular weight of 2,000 to 20,000 to form a water-insoluble composite of cationic magnetic fine particle-virus-polyacrylic acid-polyethylene glycol; and a step of forming pellets of the composite by magnetic collection and removing the resultant supernatant. Furthermore, in the above [19], there may be utilized a process for detecting a virus, which comprises a step of mixing water-soluble cationic magnetic particles where a water-soluble dextran magnetite is covalently combined through reductive amination with polyethyleneimine with a blood component containing the virus to form a water-soluble combined body of cationic magnetic fine particle-virus; a step of mixing the combined body with an aqueous solution of polyacrylic acid to form a water-soluble combined body of cationic magnetic fine particle-virus-polyacrylic acid; a step of further mixing the combined body with an aqueous solution of polyethylene glycol to form a water-insoluble composite of cationic magnetic fine particle-virus-polyacrylic acid-polyethylene glycol; a step of forming pellets of the composite by magnetic collection and removing the resultant supernatant; a step of dispersing the pellets in a liquid; and a step of denaturing the virus to release envelope proteins, capsid proteins, and nucleic acids of the virus. In addition, in the above [19], there may be utilized a process for detecting hepatitis B virus, which comprises a step of treating a water-soluble dextran magnetite with a periodic acid to form a dextran magnetite having an aldehyde and then covalently bonding it through reductive amination with polyethyleneimine having an average molecular weight of 1,800, which is a polycation, to form a water-soluble cationic magnetic fine particles; a step of mixing the fine particles with a serum containing hepatitis B virus to form a water-soluble combined body of cationic magnetic fine particle-hepatitis B virus; a step of mixing the combined body with an aqueous solution of polyacrylic acid having an average molecular weight of 25,000 to form a water-soluble combined body of cationic magnetic fine particle-hepatitis B virus-polyacrylic acid; a step of further mixing the combined body with an aqueous solution of polyethylene glycol having a molecular weight of 6,000 to form a water-insoluble composite of cationic magnetic fine particle-hepatitis B virus-polyacrylic acid-polyethylene glycol; a step of forming pellets of the composite by magnetic collection and removing the resultant supernatant; a step of dispersing the pellets in a PCR reaction solution, a step of denaturing hepatitis B virus by heating and releasing envelope proteins, capsid proteins, and nucleic acids of hepatitis B virus.

In this regard, the nucleic acid is preferably DNA or RNA. Moreover, there may be utilized a method for detecting a virus wherein the DNA of the virus obtained by the above-described methods is amplified by a nucleic acid amplification reaction. Furthermore, there may be utilized a method for detection using the envelop protein of the virus obtained by the above-described methods. In addition, there may be utilized a method for detection using the capsid protein of the virus obtained by the above-described methods. Also, there may be utilized a method for collecting and detecting a virus nucleic acid using water-soluble magnetic fine particles, masking agent, and aggregating agent described in any of them by means of an apparatus equipped with a magnetic separation mechanism.

The term “cationic functional group” in the invention means a functional group which charges positive in a protic solvent such as water and there may be, for example, exemplified a structure having a primary amino group, a secondary amino group, a tertiary amino group, a quaternary ammonium group, or an imino group.

The term “magnetic components” in the invention is a component capable of magnetic collection in response to an external magnetic field and there may be mentioned metals such as nickel, cobalt, and iron, metal oxides such as ferrite, and latex magnetic beads wherein a metal or metal oxide is dispersed in a polymer such as polystyrene. A “magnetic component” having a small size to some extent (about 100 nm) is observed not to respond the external magnetic field but this is because fluctuation due to influence of Brownian motion is larger than the response to the external magnetic field.

The term “acid structure” in the invention means a structure which charges negative in a protic solvent such as water and there may be exemplified structures of carboxylic acids, phosphoric acid, sulfuric acid, and boric acid, which may be expressed in different word as an “anionic structure”.

The term “masking agent” in the invention is a substance having a functioning group capable of neutralizing the negative charge of the water-soluble cationic magnetic fine particles and is a substance containing the “acid structure” or salt thereof in the structure.

The term “aggregating agent” in the invention is a substance having a function of changing water-soluble cationic magnetic fine particles or a water-soluble cationic magnetic fine particle-masking agent combined body into a water-insoluble aggregate through mixing with the particles or combined body, and a substance having a polyether framework such as polyethylene glycol may be exemplified. In addition, there may be preferably used alcohols such as methanol, ethanol, n-propanol, and i-propanol, ketone compounds such as acetone and methyl ethyl ketone, amide compounds such as N,N-dimethylformamide, N,N-dimethylacetamide, and N-methylolpyrrolidone, dimethyl sulfoxide, and 1,4- or 1,3-dioxane which are organic solvents miscible with water in any ratios to form a homogeneous solution.

The terra “phospholipid vesicle” in the invention means a structural substance covered with phospholipid bilayer membranes and there may be exemplified animal cells, vegetal cells, fungi, real fungi, and viruses.

The term “pellet” in the invention means a floc formed by concentrating compact cluster in a suspension at one site by conducting an operation such as centrifugation on the suspension. A floc formed by concentrating a magnetic component at one site is also defined as a “pellet”.

The term “aqueous two-phase partition” in the invention is a method of extracting a third component without using any organic solvent by mixing two substances for example, polyvinyl alcohol and an aqueous solution of polyethylene glycol utilizing difference in partition coefficient of the third component between individual layers of a solid layer and an aqueous layer formed.

According to the invention, a phospholipid vesicle such as a virus can be rapidly separated (concentrated, roughly purified) and good detection (diagnosis) results can be obtained. Moreover, according to the invention, the above operations can be automated.

### BRIEF DESCRIPTION OF THE DRAWINGS

FIG. 1 is a graph showing results of linearity confirmation test of amount of virus added and amount of virus detected.

FIG. 2 is a graph showing results of amount of virus added and absorbance of internal standard amplicon.

FIG. 3 is a graph showing results of amount of virus added and absorbance of virus DNA amplicon.

FIG. 4 is a graph comparing detected values by respective methods of Example 1 and Comparative Example 1.

FIG. 5 is a figure typically showing an automatic detection apparatus.

FIG. 6 is a graph showing results of comparison of amount of virus added and amount of virus detected.

FIG. 7 is a graph comparing amount of virus added and absorbance of internal standard DNA amplicon.

FIG. 8 is a graph comparing amount of virus added and absorbance of virus DNA amplicon.

### DETAILED DESCRIPTION OF THE INVENTION

The following will describe the invention further in detail.

The water-soluble cationic magnetic fine particles of the invention contain a substance having a cationic functional group, a substance having a hydroxyl group, and a substance having magnetism (it is sometimes referred to as a magnetic component). The form contained is not particularly limited but it is preferable that the substance having a cationic functional group (substance having a functional group exhibiting a cationic character in an aqueous solution) is immobilized into magnetic fine particles which are composites of substance having a hydroxyl group-magnetic component. In the water-soluble magnetic fine particles of the invention, the substance having a hydroxyl group preferably has a polyol framework.

The substance having a hydroxyl group is desirably a polymer having a polyol framework in the structure.

As the polyol, there may be mentioned polysaccharides such as dextran, dextrin, cellulose, agarose, starch, and gelan; polysaccharide derivatives such as carboxymethyl cellolose, diethylamino cellulose, hydroxyacetyl cellulose, hydroxyacetyl cellulose, carboxymethyl dextran, diethylaminoethyl cellulose, and diethylaminoethyl dextran; synthetic polyols such as polyvinyl alcohol and polyallyl alcohol; polymers polymerized from at least one compound of polymerizable monomers having a hydroxyl group, such as allyl alcohol, 2-hydroxyethyl(meth)acrylate, glycerol-mono(meth)acrylate, and 2-hydroxyethyl(meth)acrylamide as a polymerizable monomer; polyvinyl alcohol random copolymers obtained by deprotection of hydroxyl group from polymers polymerized from at least one compound of polymerizable monomers containing vinyl alcohol having an acetate ester-type, trimethylsilyl ether-type, or t-butyloxycarbonyloxy-type protected hydroxyl group as a polymerizable monomer. These polyols may be used singly or in combination of two or more thereof.

Of these, neutral polymers containing a sugar skeleton, such as polysaccharides or polysaccharide derivatives are preferable. Specifically, the substance may be a compound having an action of forming a phase for the aqueous two-phase partition and there may be mentioned a water-soluble polymer containing a sugar skeleton such as glucose skeleton, for example, starch, more preferably dextran. As dextran, one having an optimum weight-average molecular weight may be selected by experiment and used. For example, one having a weight-average molecular weight of 10,000 to 100,000, one having a weight-average molecular weight of 60,000 to 600,000, and also one having a weight-average molecular weight of 67,300 to 500,900 may be mentioned, which may be available from Sigma, for example.

In the water-soluble cationic magnetic fine particles, the magnetic component contained in the substance having a hydroxyl group-magnetic component composite is, for example, magnetic fine particles and there may be mentioned magnetic metal fine particles and magnetic oxide fine particles. These magnetic fine particles may contain a rare-earth element or a transition metal element, if necessary. As the magnetic metal fine particles, there may be, for example, mentioned metal fine particles such as Fe—Co, Fe—Ni, Fe—Al, Fe—Ni—Al, Fe—Co—Ni, Fe—Ni—Al—Zn, and Fe—Al—Si. As the magnetic oxide fine particles, there may be mentioned iron oxide (ferrite)-type ferromagnetic fine particles represented by FeOₓ (4/3≦x≦3/2) and ferrite wherein part of Fe is partially substituted by Ni or Co. More specifically, as materials of the magnetic fine particles, there may be mentioned fine particles of magnetite, nickel oxide, ferrite, cobalt iron oxide, barium ferrite, carbon steel, tungsten steel, KS steel, rare-earth cobalt magnet, maghemite, hematite, and gesite. The shape of these magnetic fine particles may be any of spherical, needle-like, spindle-shaped, and amorphous ones.

As a pretreatment for introducing the substance having a hydroxyl group or the substance having a cationic functional group, the above magnetic fine particles may be subjected to surface treatment. As the surface treatment, a silane-based coupling treatment, a titanium-based coupling treatment, a phosphoric acid-based coupling treatment, an acid treatment with hydrochloric acid or sulfuric acid, or an alkali treatment with sodium hydroxide or the like may be conducted.

In the case that the period until the precipitate of the magnetic fine particles can be confirmed magnetic metal fine particles may be so short as about 30 seconds, the average particle size is from 1 nm to 10 μm. The above magnetic fine particles are homogeneously dispersed in an aqueous solution and precipitate is preferably not formed for a long time The average particle size is preferably from 1 nm to 300 nm.

Moreover, the above magnetic fine particles may be magnetic component whose surface is coated with a latex such as polystyrene or polymethyl(meth)acrylate or a magnetic component (they are called latex magnetic beads) wherein the above magnetic fine particles are dispersed in latex beads. The average particle size of the latex magnetic beads is preferably from 20 nm to 300 nm.

With regard to the substance having a hydroxyl group-magnetic component composite, as the composite mode of the above polyol and the above magnetic component, physical adsorption and covalent bond formation may be mentioned.

Moreover, as the substance having a hydroxyl group-magnetic component composite, ferrite fine particles coated with a polyol obtained by coprecipitation method of adding an alkali such as ammonia or sodium hydroxide to an aqueous iron ion solution containing the polyol may be used (e.g., cf. JP-A-6-92640). More specifically, as described in U.S. Pat. No. 4,452,773, it can be obtained by adding a mixed aqueous solution (10 ml) of ferric chloride hexahydrate (1.51 g) and ferrous chloride tetrahydrate (0.64 g) to a 50% by weight aqueous solution (10 ml) of dextran, stirring the whole, and adding a 7.4% by volume aqueous ammonia solution dropwise thereto under heating in a water bath at 60 to 65° C. so that the pH becomes from about 10 to 11, whereby a reaction is effected for 15 minutes.

With regard to the water-soluble cationic magnetic fine particles to be used in the invention, as the composite mode of the above magnetic fine particles prepared by the above method and the above substance having a cationic functional group, physical adsorption and covalent bond formation may be mentioned.

More specifically, an aqueous solution (1% by weight, 100 mL) of the dextran-coated magnetic fine particles is treated with sodium periodate (10 mg), the whole is reacted at 50° C. for 5 hours to form a dextran-coated magnetic fine particles. Then, an aqueous solution wherein polyethyleneimine (M.W.=1800, 1 g) is dissolved in ultrapure water (9 g) is added thereto and the whole is stirred for 14 hours to form a dextran coating wherein polyethyleneimine is combined via an imine bond and then an aqueous solution wherein sodium borohydride (10 mag) is dissolved in ultrapure water (1 mL) is added and stirred for 24 hours to convert the imine bond to an amine bond. By the method described above, a dextran-coated magnetic fine particles into which polyethyleneimine is immobilized can be obtained.

As another method, an aqueous solution (1% by weight, 10 mL) of magnetic fine particles having a glycidyl group obtained by reacting magnetic fine particles with glycidyloxypropyltriethoxysilane or reacting polyvinyl alcohol-coated magnetic fine particles with epichlorohydrin under alkaline conditions is mixed with ε-polylysine (100 mg) and the whole is stirred for 24 hours, whereby polylysine-immobilized magnetic fine particles can be obtained.

Moreover, the cationic magnetic fine particles may be also obtained by reacting the hydroxyl group on the magnetic fine particles with an amine-introducing reagent such as N,N-diethylaminoethyl chloride hydrochloride (DEAE-Cl.HCl). More specifically, DEAE-Cl.HCl (100 mg) and 1N aqueous sodium hydroxide solution (1 mL) are added to an aqueous dextran-coated magnetic fine particle solution (1% by weight, 10 mL) and the whole is reacted for 24 hours, whereby an aqueous DEAE-substituted dextran-coated magnetic fine particle solution is obtained.

As a property required for the water-soluble cationic magnetic fine particles to be used in the invention, the cationic magnetic fine particles preferably have positive charge. The charge of the water-soluble cationic magnetic fine particles can be measured as ξ potential and, for example, ELS-800 (manufactured by Otsuka electronics) or the like may be used as a measuring device. The ξ potential of the cationic magnetic fine particles is preferably 0 eV or higher, more preferably +5 eV or higher, further preferably +15 eV or higher, and most preferably +30 eV or higher. As a qualitative form-confirming method, there may be adopted a method of confirming change of a liquid from brown to colorless transparent by mixing an aqueous solution of cationic magnetic fine particles with CM cellulofine C-500-sf (name of article, manufactured by Chisso Corporation), followed by mixing.

As a preferable property required for the water-soluble cationic magnetic fine particles to be used in the invention, the average particle size of the magnetic fine particles is from 1 nm to 1000 nm, preferably from 1 to 500 nm, more preferably from 10 to 300 nm, and further preferably from 30 to 150 nm.

As a preferable property required for the water-soluble cationic magnetic fine particles to be used in the invention, a homogeneous dispersion may be mentioned at the virus-trapping operation. In the case that aggregation occurs in the aqueous solution of the water-soluble cationic magnetic fine particles, the solution may be used after re-dispersion thereof by stirring, ultrasonic treatment, or heating. After the above operation, it is desirable that the substance containing a hydroxyl group having magnetism is stably homogeneously dispersed as an aqueous solution without aggregation and precipitation for 1 minute or more. It is preferable that aggregation and precipitation are not generated preferably for 2 weeks or more, more preferably for 6 months or more.

The change with time can be confirmed, for example, by charging an aqueous solution of the substance containing a hydroxyl group having magnetism into a transparent sample vial, allowing it to stand usually under a temperature condition of ordinary temperature, preferably from 4° C. to 37° C., and visually observing the generation of precipitate every a constant period or conducting a magnetic collection operation within 10 seconds.

In the case that the water-soluble cationic magnetic fine particles are homogeneously dispersed in an aqueous solution, the aqueous solution behaves as a magnetic fluid even when magnetic collection operation is conducted, and the particles are preferably not magnetically collected. On the other hand, in the case that precipitation has been generated, the resultant precipitate is instantaneously collected under the above conditions, so that the confirmation can be easily performed. By such an operation, change with time of the water-soluble cationic magnetic fine particles can be confirmed.

In the invention, for the collection of a virus, it is preferable to use magnetic fine particles wherein a substance having a cationic functional group is used. However, there may be suitably used magnetic fine particles to which various antibodies capable of recognizing envelope proteins of phospholipid vesicles are combined.

In the invention, as a procedure for enabling the collection of the water-soluble cationic magnetic fine particles, there is employed an aggregating agent which forms a molecular complex with the polyol of the magnetic fine particles to form an aggregate.

In the invention, the aggregating agent is a substance capable of forming a molecular complex with the above water-soluble magnetic fine particles. For example, there may be mentioned a substance having a polyalkylene glycol structure and specifically, polyethylene glycol, polypropylene glycol, polyethylene glycol-polypropylene glycol random copolymer, and polyethylene glycol-polypropylene glycol block copolymer.

In addition, as other embodiment of the invention, there may be mentioned polymethoxyethoxy(meth)acrylate, poly(diethylene glycol-(meth)acrylate-methyl ether) poly(triethylene glycol-(meth)acrylate-methyl ether), poly(tetraethylene glycol-(meth)acrylate-methyl ether), poly(polyethylene glycol(meth)acrylate), and random and block copolymers thereof, or poly(glycerin-2-ethyl ether), poly(glycerin-2-diethylene glycol methyl ether), poly(glycerin-2-triethylene glycol methyl ether), poly(glycerin-2-tetraethylene glycol methyl ether), poly(glycerin-2-polyethylene glycol ether), poly(glycerin-2-polypropylene glycol ether), and a poly(glycerin-2-polyethylene glycol ether)(glycerin-2-polypropylene glycol ether) copolymer.

Of these, the “polyalkylene glycol” may be any one as far as it has an action of forming a phase for aqueous two-phase partition. There may be mentioned one forming a phase for the partition in combination with a more hydrophilic polymer or more hydrophobic polymer. The polyalkylene glycol is water-soluble and the most suitable one can be determined by experiments and can be selectively used. It is preferably polyethylene glycol (PEG) or polypropylene glycol, and more preferably polyethylene glycol. As the polyethylene glycol, one having the most suitable molecular weight can be selected by experiments and be used and there may be mentioned those having a number-average molecular weight ranging from about 200 to 25,000, preferably a number-average molecular weight ranging from about 3,000 to 20,000, more preferably a number-average molecular weight ranging from about 6,000 to 15,000, and further preferably a number-average molecular weight ranging from about 8,000 to 10,000, which are available, for example, from Sigma, Wako pure Chemical Industries, Ltd., and the like.

The aggregating agent can be used in a powder form as it is but is preferably used as an aqueous solution. In the latter case, the concentration of the aggregating agent is preferably 30% by weight or less. In the case of higher concentration, the solution becomes difficult to handle since viscosity is too high and thus, the problem is particularly serious when a small amount thereof is to be taken out. In the case that the aggregating agent is necessarily used as a powder, for example, increased concentration of the aggregating agent is necessary for the formation of an aggregate with the substance having a hydroxyl group, it is desirable to utilize a powder obtained by freeze-drying from water.

The aggregating agent in the invention means a substance having a function of forming a water-insoluble aggregate having magnetic responsibility by mixing with water-soluble cationic magnetic fine particles or a water-soluble composite containing cationic magnetic fine particles and is not particularly limited the above substance groups.

In the invention, as the masking agent, there may be mentioned a substance containing an acid structure selected from the group consisting of carboxylic acids, phosphoric acid, sulfuric acid, and boric acid. Specifically, there may be mentioned poly(meth)acrylic acid, polycarboxymethylstyrene, hyaluronic acid, α-polyglutamic acid, ω-polyglutamic acid, gelan, carboxymethyl cellulose, carboxymethyl dextran, polyphosphoric acid, poly(phosphoric acid sugar), nucleic acids, phosphoric acid, citric acid, dextran sulfuric acid, polystyrylsulfuric acid, and polystyrylboric acid. The masking agent is preferably a poly(meth)acrylic acid, a nucleic acid, or polyphosphoric acid, more preferably a poly(meth)acrylic acid having an average molecular weight of 10,000 to 100,000, and further preferably a poly(meth)acrylic acid having an average molecular weight of 25,000 to 50,000.

The masking agent in the invention is capable of neutralizing the positive charge of the magnetic fine particles or converting it into magnetic fine particles having negative charge through combination with the amino group present on the surface of the cationic magnetic fine particles to form anion complex and thus the masking agent may be called a neutralizing agent or a surface-modifying agent. Substances having such a function can be used as the masking agent, which is not particularly limited to the above substance groups.

In the invention, the water-soluble combined body of cationic magnetic fine particle-phospholipid vesicle can be formed by mixing cationic magnetic fine particles with a phospholipid vesicle. As mixing methods usable in the invention, there may be mentioned stirring with a magnetic stirrer, stirring with a mechanical stirrer, mixing with a vortex mixer, mixing with tapping the tube, mixing with pipetting, and the like but the method is not particularly limited thereto. The time required for the stirring depends on a stirring method but is 10 seconds or more, preferably 20 seconds or more, more preferably 30 seconds or more at 1,000 rpm in the case that 60 μm of a liquid present in a 1.5 mL screw-cap tube.

In the invention, the water-insoluble composite of cationic magnetic fine particles-phospholipid vesicle-aggregating agent can be formed by adding an aggregating agent to a liquid containing water-soluble cationic magnetic fine particles and a phospholipid vesicle and mixing the whole by an appropriate method. As mixing methods usable in the invention, there may be mentioned stirring with a magnetic stirrer, stirring with a mechanical stirrer, mixing with a vortex mixer, mixing with tapping the tube, mixing with pipetting, and the like but the method is not particularly limited thereto. The time required for the stirring depends on a stirring method but is 10 seconds or more, preferably 20 seconds or more, more preferably 30 seconds or more at 1,000 rpm in the case that 80 μm of a liquid present in a 1.5 mL screw-cap tube.

The amount of the aggregating agent to be added is preferably from 0.1 to 20% by weight as a dry weight relative to a mixed liquid of the cationic magnetic fine particles, the phospholipid vesicle, and the aggregating agent. Particularly preferable is a case that the aggregating agent is from 4 to 10% by weight. The operation may be conducted at room temperature but may be conducted under ice-cooling, if necessary.

In the invention, as a method for separating the composite of cationic magnetic fine particles-phospholipid vesicle-aggregating agent from the liquid, there may be mentioned pelletization by magnetic separation, pelletization by centrifugation and removal of a supernatant, pelletization through liquid removal by filtration, and the like. The operation may be conducted at room temperature but may be conducted under ice-cooling, if necessary.

Moreover, the magnetic pellets obtained by the above operation can be subjected to a latex aggregation operation using antibody-immobilized latex magnetic beads after re-dispersed into a buffer containing physiological saline.

In the case that the phospholipid vesicle is a blood virus, it can be roughly purified by the following method.

In the invention, the water-soluble composite of cationic magnetic fine particle-virus is formed by mixing cationic magnetic fine particles with a plasma or serum containing the virus. As mixing methods usable in the invention, there may be mentioned stirring with a magnetic stirrer, stirring with a mechanical stirrer, mixing with a vortex mixer, mixing with tapping the tube, mixing with pipetting, and the like but the method is not particularly limited thereto. The time required for the stirring depends on a stirring method but is 10 seconds or more, preferably 20 seconds or more, more preferably 30 seconds or more at 1,000 rpm in the case that 120 μm of a liquid present in a 1.5 mL screw-cap tube.

In the invention, the water-soluble composite of cationic magnetic fine particle-virus-masking agent is formed by mixing cationic magnetic fine particles, a plasma or serum containing the virus, and a masking agent. As mixing methods usable in the invention, there may be mentioned stirring with a magnetic stirrer, stirring with a mechanical stirrer, mixing with a vortex mixer, mixing with tapping the tube, mixing with pipetting, and the like but the method is not particularly limited thereto. The time required for the stirring depends on a stirring method but is 60 seconds or more, preferably 60 seconds or more, more preferably 120 seconds or more at 1,000 rpm in the case that 120 μm of a liquid present in a 1.5 mL screw-cap tube.

In the invention, the water-insoluble composite of a cationic magnetic fine particles-phospholipid vesicle-aggregating agent is formed by adding an aqueous solution of an aggregating agent to the water-soluble combined body of cationic magnetic fine particle-virus-masking agent and mixing the whole by an appropriate method. As mixing methods usable in the invention, there may be mentioned stirring with a magnetic stirrer, stirring with a mechanical stirrer, mixing with a vortex mixer, mixing with tapping the tube, mixing with pipetting, and the like but the method is not particularly limited thereto. The time required for the stirring depends on a stirring method but is 10 seconds or more, preferably 20 seconds or more, more preferably 30 seconds or more at 1,000 rpm in the case that 80 μm of a liquid present in a 1.5 mL screw-cap tube.

The amount of the aggregating agent to be added is preferably from 0.1 to 10% by weight as a dry weight relative to a mixed liquid of the plasma or serum, the cationic magnetic fine particles, and the aggregating agent. Particularly preferable is a case that the aggregating agent is from 4 to 10% by weight. The operation may be conducted at room temperature but may be conducted under ice-cooling, if necessary.

In the invention, as a method for collecting the composite obtained, there may be mentioned pelletization by magnetic separation, pelletization by centrifugation and removal of a supernatant, pelletization through liquid removal by filtration, and the like. The operation may be conducted at room temperature but may be conducted under ice-cooling, if necessary.

In the invention, the magnetic separation of the aggregate is desirably effected by arranging magnet on the side surface of the vessel in which an aggregate suspension to be subjected to magnetic separation conditions is placed. The vessel herein is an Eppendorf tube, a screw-cap tuber a PCR tube, or the like. Moreover, the vessel may have a structure having a liquid-draining mouth at the bottom capable of simple and convenient charge and discharge of liquid, such as a pipette tip. As another embodiment of the invention, the aggregate may be collected by directly dipping a magnet in the vessel in which the aggregate suspension is placed or dipping a coated article into the liquid so that a magnet does not come into contact with the suspension.

The magnetic collection is completed at the time when brown color derived from the magnetic fine particles is not confirmed from a supernatant of magnetic separation. In the case that the magnetic fine particles are contained in an amount of 0.06% by weight as a dry weight relative to the aggregate suspension, the time required for the magnetic collection is within about 5 minutes Increase in an amount of the magnetic fine particles contained in the liquid containing the aggregate enables shortening of the time required for the magnetic separation. Moreover, decrease in the distance for the magnetic separation, specifically, magnetic separation from a side surface of a vessel having a narrow width with a magnet enables shortening of the time required for the magnetic separation.

As the other embodiment of the invention, using the above vessel having a hole capable of charging and discharging a liquid at the bottom, the supernatant can be removed simultaneously to magnetic separation by discharging the liquid simultaneously with the magnetic separation.

In the invention, the removal of the aggregate may be conducted by removing the supernatant simultaneously with magnetic separation as described above or by carefully removing the supernatant using a pipette or the like so as not to such pellets after the pellets are formed. At this time, the supernatant-removing operation is desirably conducted under the conditions for the magnetic separation as they are and, after the removal of the supernatant, a liquid leaked out from the pellets is also desirably removed.

After the magnetic pellets obtained by the above operation is re-dispersed in, for example, Ampdirect (trade name, manufactured by Shimadzu Corporation), they are mixed with a PCR reaction solution and then nucleic acid amplification can be carried out. Moreover, as the other embodiment of the invention, after a virus is denatured by a method usually conducted by those skilled in the art, for example, re-dispersing the virus in an aqueous solution of a chaotropic salt such as guanidine hydrochloride, the denatured virus is brought into contact with a support having a silanol structure on the surface, such as a glass filter or silica beads to adsorb a nucleic acid, an elution operation from the support is conducted, and then the nucleic acid is mixed with a PCR reaction solution, whereby nucleic acid amplification can be effected.

Furthermore, the magnetic pellets obtained by the above operation can be subjected to latex aggregation operation using latex magnetic beads to which an antibody is immobilized after re-dispersed in a buffer containing physiological saline.

The following will specifically describe further in detail one example of a manual method for virus collection of the invention in the combination of hepatitis B virus with polyethyleneimine-immobilized dextran-coated magnetic fine particles.
1) An aqueous divalent and trivalent iron chloride solution is mixed in the presence of dextran and ammonia is added thereto to thereby prepare dextran-coated magnetic fine particles capable of being homogeneously dispersed in water.
2) The dextran-coated magnetic fine particles is reacted with sodium periodate to form dextran-coated magnetic fine particles having an aldehyde group, polyethyleneimine is mixed to prepare a polyethyleneimine-immobilized dextran-coated magnetic fine particles which are bonded through an imine bond, sodium borohydride is added to reduce the imine bond into an amine bond to prepare a polyethyleneimine-immobilized dextran-coated magnetic fine particles.
3) A plasma or serum of a subject to be tested who is expected to be infected with hepatitis B virus is mixed with the polyethyleneimine-immobilized dextran-coated magnetic fine particles, followed by stirring for 30 seconds.
4) An aqueous polyacrylic acid solution is added, followed by stirring for 2 minutes.
5) An aqueous polyethylene glycol solution is added, followed by stirring for 30 seconds.
6) Magnetic separation is conducted using neodymium magnet to form pellets.
7) A supernatant is removed using a pipette.
8) Ampdirect (manufactured by Shimadzu Corporation) is added and the whole is stirred to homogeneously disperse the pellets.
9) The whole is heated at 95° C. for 5 minutes using Heat Block( manufactured by TITEC).
10) The dispersion is mixed with a nucleic acid-amplifying reagent of AMPLICORE HBM (Roche Diagnostics) and a thermal cycler is set according to the method described in the procedure manual of AMPLICORE HBM to amplify nucleic acids.
11) Detection is conducted according to the method described in the procedure manual of AMPLICORE HBM.

The following will specifically describe further in detail one example of an automatic method for virus collection of the invention in the combination of hepatitis B virus with polyethyleneimine-immobilized dextran-coated magnetic fine particles.
1) The magnet unit of an automatic nucleic acid-extracting apparatus MP12 (manufactured by Precision System Science) is replaced by a magnet unit where 13 pieces of a magnet of 28 mm×4 mm×8 mm obtained by stacking 7 pieces of a neodymium magnet of 4 mm×4 mm×8 mm are mounted in series.
2) The polyethyleneimine-immobilized dextran-coated magnetic fine particles are placed in a second reaction lane of MP12.
3) An aqueous polyacrylic acid solution is placed in the third lane of a reaction tray of MP12.
4) A aqueous polyethylene glycol solution for aggregation is placed in the fourth lane of a reaction tray of MP12.
5) An aqueous polyethylene glycol solution for washing is placed in the fifth lane of a reaction tray of MP12.
6) Ampdirect (trade name, manufactured by Shimadzu Corporation) is placed in the sixth lane of a reaction tray of MP12.
7) The reaction trays containing liquid of 2) to 5), a 1.5 mL screw-cap tube containing a plasma or serum of a subject containing hepatitis B virus, and a tip for Binding/Free separation are provided on MP12.
8) A plasma or serum of a subject to be tested who is expected to be infected with hepatitis B virus is mixed with the polyethyleneimine-immobilized dextran-coated magnetic fine particles, followed by pipetting for 60 seconds.
9) An aqueous polyacrylic acid solution is added, followed by pipetting for 2 minutes.
10) An aqueous polyethylene glycol solution for aggregation is added, followed by pipetting for 60 seconds.
11) Magnetic separation is conducted in the tip to form pellets.
12) A liquid separated from the pellets is discharged and removed from the tip.
13) An aqueous polyethylene glycol solution for washing is sucked and discarded.
14) Ampdirect (manufactured by Shimadzu Corporation) is sucked and pipetted to homogeneously disperse the pellets.
15) The resultant pellet dispersion is transferred into a heat block of MP12 set at 105° C.
16) The liquid subjected to the heat treatment is transferred into the first lane of the reaction tray.
17) The liquid is mixed with a nucleic acid-amplifying reagent of AMPLICORE HBM (Roche Diagnostics) and a thermal cycler is set according to the method described in the procedure manual of AMPLICORE HBM to amplify nucleic acids.
18) Detection is conducted according to the method described in the procedure manual of AMPLICORE HBM.

### EXAMPLES

The following will illustrate the invention with reference to Examples but the invention is not limited to these Examples.

Among the reagents used in the investigation, the following were prepared as follows.

Preparation of Aqueous Polyethylene Glycol Solution

Preparation of Aqueous Polyethylene Glycol Solution

Polyethylene glycol (6 KDa, 2.5 g), ultrapure water (97.5 g), and diethyl pyrocarbonate (0.1 mL) were added to a 150 mL glass bottle equipped with a magnetic stirrer bar and then the bottle was capped, followed by stirring at room temperature overnight. Sterilization was conducted in an autoclave under conditions of 120° C. and 40 minutes.

Preparation of Aqueous Iron Chloride Solution

Ferric chloride hexahydrate (81.9 g), ferric chloride tetrahydrate (29.8 g), and ultrapure water (188.3 g) were placed in a 5000 mL beaker equipped with a magnetic stirrer bar and then the whole was stirred with nitrogen bubbling at room temperature for 2 hours to thereby achieve homogeneous dissolution. The resultant solution was filtrated by suction under reduced pressure and the resultant yellow-brown liquid was measured up to 300 mL. In this regard, as the ultrapure water, Direct-Q (trade name) manufactured by Millipore was used for the preparation.

Preparation of Aldehyde Group-Modified Dextran Magnetite

An aqueous 5.0 by weight solution (1 L) of dextran (manufactured by Wako Pure Chemicals Ltd., 40 KDa) was placed in a 2 L three-neck flask equipped with a mechanical stirrer, a reflux column, and a nitrogen line, followed by heating at 65° C. under stirring. The above aqueous iron chloride solution (100 mL) was added dropwise thereto and, after completion of the dropwise addition, the whole was stirred for 10 minutes. Then, the whole was further stirred for 30 minutes while an aqueous 28% by weight ammonia solution was added dropwise so that the pH becomes about 10 to 11. The solution was filtrated by suction under reduced pressure. Two cycles of a dialysis operation using ion-exchange water (5 L) were conducted against part of the resultant filtrate (100 mL), where one cycle included four times of 3-hour dialysis and one time of 12-hour dialysis. Through the operation, there were obtained magnetic fine particles which have an average particle size of 102±15.4 nm and to which dextran was coated.

In order to prepare a dextran-coated magnetite having an aldehyde group, the aqueous dextran-coated magnetite solution (400 mL) prepared by the above method was added to a 500 mL three-neck flask equipped with a mechanical stirrer, a reflux column, and a nitrogen line, and then sodium periodate (40 mg) dissolved in ultrapure water (10 mL) was added thereto, followed by heating at 50° C. for 5 hours and cooling to room temperature.

The magnetic fine particles were used in next reaction without particular purification. The average particle size was 110±15.7 nm.

Preparation of ε-Polylysine-Immobilized Dextran-Coated Magnetite

In order to prepare a ε-polylysine-immobilized dextran-coated magnetite, the aqueous solution (100 mL) of dextran-coated magnetite having an aldehyde group prepared by the above method was added to a 200 mL three-neck flask equipped with a mechanical stirrer, a reflux column, and a nitrogen line, and then ε-polylysine (1 g) dissolved in ultrapure water (9 g) was added thereto at 20° C., followed by stirring for 14 hours. Separately, a foamed solution obtained by dissolving sodium borohydride (20 mg) in ultrapure water (1 mL) placed in an 8 mL test tube was added to the above flask. A 20 mL eggplant-shaped flask was capped with a cotton stopper, followed by stirring for 24 hours. The solution was filtrated by suction under reduced pressure. Two cycles of a dialysis operation using ion-exchange water (5 L) were conducted against the resultant filtrate, where one cycle included four times of 3-hour dialysis and one time of 12-hour dialysis. The average particle size of the particles obtained by the operation was 112±37.6 nm.

Preparation of Polyethyleneimine 1800-Immobilized Dextran-Coated Magnetite

In order to prepare a polyethyleneimine 1800-immobilized dextran-coated magnetite, the aqueous solution (100 mL) of dextran-coated magnetite having an aldehyde group prepared by the above method was added to a 200 mL three-neck flask equipped with a mechanical stirrer, a reflux column, and a nitrogen line, and then polyethyleneimine (M.W.=1,800, 1 g) dissolved in ultrapure water (9 g) was added thereto at 20° C., followed by stirring for 14 hours. Separately, a foamed solution obtained by dissolving sodium borohydride (20 mg) in ultrapure water (1 mL) placed in an 8 mL test tube was added to the above flask. A 20 mL eggplant-shaped flask was capped with a cotton stopper, followed by stirring for 24 hours.

The solution was filtrated by suction under reduced pressure. Two cycles of a dialysis operation using ion-exchange water (5 L) were conducted against the resultant filtrate, where one cycle included four times of 3-hour dialysis and one time of 12-hour dialysis. The average particle size of the particles obtained by the operation was 118±21.5 nm.

Preparation of Polyethyleneimine 10000-Immobilized Dextran-Coated Magnetite

In order to prepare a polyethyleneimine-immobilized dextran-coated magnetite, the aqueous solution (100 mL) of dextran-coated magnetite having an aldehyde group prepared by the above method was added to a 200 mL three-neck flask equipped with a mechanical stirrer, a reflux column, and a nitrogen line, and then polyethyleneiminie (M-W.=10,000, 1 g) dissolved in ultrapure water (9 g) was added thereto at 20° C., followed by stirring for 14 hours. Separately, a foamed solution obtained by dissolving sodium borohydride (20 mg) in ultrapure water (1 mL) placed in an 8 mL test tube was added to the above flask. A 20 mL eggplant-shaped flask was capped with a cotton stopper, followed by stirring for 24 hours.

The solution was filtrated by suction under reduced pressure. Two cycles of a dialysis operation using ion-exchange water (5 L) were conducted against the resultant filtrate, where one cycle included four times of 3-hour dialysis and one time of 12-hour dialysis. The average particle size of the particles obtained by the operation was 118±21.5 nm.

Masking Agent 1: Preparation of Masking Agent Using Polyacrylic Acid 2500

Polyacrylic acid (M.W.=25,000, 250 mg), ultrapure water (99.75 g), and diethyl pyrocarbonate (0.1 mL) were added to a 150 mL glass bottle equipped with a magnetic stirrer bar and then the bottle was capped, followed by stirring at room temperature all day and night. Sterilization was conducted in an autoclave under conditions of 120° C. and 40 minutes.

Masking Agent 2: Preparation of Masking Agent Using Polyacrylic Acid 5000

Polyacrylic acid (M.W.=50,000, 250 mg), ultrapure water (99.75 g), and diethyl pyrocarbonate (0.1 mL) were added to a 150 mL glass bottle equipped with a magnetic stirrer bar and then the bottle was capped, followed by stirring at room temperature all day and night. Sterilization was conducted in an autoclave under conditions of 120° C. and 40 minutes.

Clinical Specimen—HBV Positive Human Normal Plasma

Blood taken from a hepatitis B patient was processed to prepare 41 specimens of a sample as HBV positive human normal plasma.

For Linearity Test—HBV Positive Human Normal Plasma—10⁵ Copies/mL

A human normal plasma containing hepatitis B virus of 10⁶ copies/mL (300 μL, amount of hepatitis B virus was confirmed using AMPLICORE HBM) was mixed with an HBV negative human normal plasma (2700 μL) and the whole was stirred for 10 seconds using a vortex mixer to form a human normal plasma containing hepatitis B virus of 10⁵ copies/mL.

For Linearity Test—HBV Positive Human Normal Plasma—10⁴ Copies/mL

A human normal plasma containing hepatitis B virus of 10⁵ copies/mL prepared above (300 μL) was mixed with an HBV negative human normal plasma (2700 μL) and the whole was stirred for 10 seconds using a vortex mixer to form a human normal plasma containing hepatitis B virus of 10⁴ copies/mL.

For Linearity Test—HBV Positive Human Normal Plasma—10³ Copies/mL

A human normal plasma containing hepatitis B virus of 10⁴ copies/mL prepared above (300 μL) was mixed with an HBV negative human normal plasma (2700 μL) and the whole was stirred for 10 seconds using a vortex mixer to form a human normal plasma containing hepatitis B virus of 10³ copies/mL.

For Linearity Test—HBV Positive Human Normal Plasma—10² Copies/mL

A human normal plasma containing hepatitis B virus of 10³ copies/mL prepared above (300 μL) was mixed with an HBV negative human normal plasma (2700 μL) and the whole was stirred for 10 seconds using a vortex mixer to form a human normal plasma containing hepatitis B virus of 10² copies/mL.

For Interference Test—Bilirubin C-Added HBV Positive Human Normal Plasma—10^{3.95} Copies/mL

A sample of bilirubin C of interference check A plus (183 mg/dL, manufactured by Sysmex Corporation) dissolved in 2 mL of ultrapure water (200 μL) was mixed with a human normal plasma containing hepatitis B virus of 10⁴ copies/mL (1800 μL) and the whole was stirred using a vortex mixer to prepare a bilirubin C (183 mg/L)-added HBV positive (10^{3.95} Copies/mL) human normal plasma.

For Interference Test-Bilirubin F—added HBV Positive Human Normal Plasma—10^{3.95} Copies/mL

A sample of bilirubin F of interference check A plus (193 mg/dL, manufactured by Sysmex Corporation) dissolved in 2 mL of ultrapure water (200 μL) was mixed with a human normal plasma containing hepatitis B virus of 10⁴ copies/mL (1800 μL) and the whole was stirred using a vortex mixer to prepare a bilirubin F (193 mg/L)-added HBV positive (10^{3.95} Copies/mL) human normal plasma.

For Interference Test-Hemolytic Hemoglobin—added HBV Positive Human Normal Plasma—10^{3.95} Copies/mL

A sample of hemolytic hemoglobin of interference check A plus (4840 mg/dL manufactured by Sysmex Corporation) dissolved in 2 mL of ultrapure water (200 μL) was mixed with a human normal plasma containing hepatitis B virus of 10⁴ copies/mL (1800 μL) and the whole was stirred using a vortex mixer to prepare a hemolytic hemoglobin (4840 mg/L)-added HBV positive (10^{3.95} Copies/mL) human normal plasma.

For Interference Test—chyle-added HBV Positive Human Normal Plasma—10^{3.95} Copies/mL

A sample of chyle of interference check A plus (18400 degree, manufactured by Sysmex Corporation) dissolved in 2 mL of ultrapure water (200 μL) was mixed with a human normal plasma containing hepatitis B virus of 10⁴ copies/mL (1800 μL) and the whole was stirred using a vortex mixer to prepare a chyle (1840 degree)-added HBV positive (10^{3.95} copies/mL) human normal plasma.

### Comparative Example 1

HBV positive human normal plasma (50 μL) and Sol-A (25 μL) were added to a 1.5 mL screw-cap tube and the whole was stirred for 30 seconds using a vortex mixer to make the analyte turbid. Using a high-speed centrifuge, pellets were formed at the bottom of the screw-cap tube under conditions of 15000 rpm and 5 minutes. The supernatant was carefully removed using a pipette. Ampdirect (manufactured by Shimadzu Corporation, 50 μL) was added to the resultant pellets and the whole was stirred for 30 seconds using a vortex mixer to disperse the pellets. The screw-cap tube was placed on a heat block at 95° C. (manufactured by Taitec, for a 1.5 mL screw-cap tube), heated for 5 minutes, and then cooled to room temperature. The above thermally treated liquid (25 μL) and a PCR reaction solution (MMX, 75 μL) of AMPLICORE HBM (manufactured by Roche Diagnostics) were mixed in a 200 μL PCR tube, then mixed by light tapping the tube, and a nucleic acid amplification reaction was carried out using a thermal cycler (9600R, manufactured by Roche Diagnostics). The thermal cycler was operated under the following conditions.

Hold . . . at 50° C. for 2 minutes

Hold . . . at 96° C. for 5 minutes

Cycles 1 to 30 . . . at 96° for 20 seconds, at 58° C. for 20 seconds, and at 72° C. for 30 seconds

Hold . . . at 72° C. for 10 minutes

Hold . . . held at 72° C.

The resultant nucleic acid-amplified product was used according to the manual together with an avidin plate and various hybridizing reagents attached to AMPLICORE HBM. In this case, Columbus 2 (manufactured by Roche Diagnostics) was used as a microplate washer. Detection was conducted using an AMPLICORE HBM-dedicated plate reader (NJ-2300, manufactured by Roche Diagnostics).

### Example 1

HBV positive human normal plasma (50 μL) and an aqueous magnetic beads solution (10 μL) were added to a 1.5 mL screw-cap tube and the whole was stirred for 30 seconds using a vortex mixer. The masking agent 1 (20 μL) was added thereto, followed by stirring for 2 minutes using a vortex mixer. Then, an aggregating agent (40 μL) was added thereto and the whole was stirred for 30 seconds using a vortex mixer to form an aggregate. Magnetic separation was conducted under conditions of neodymium magnet and 5 minutes to form pellets at side surface of the screw-cap tube. The supernatant was carefully removed using a pipette. Ampdirect (manufactured by Shimadzu Corporation, 50 μL) was added to the resultant pellets and the whole was stirred for 30 seconds using a vortex mixer to homogeneously disperse the pellets. The screw-cap tube was placed on a heat block (manufactured by Taitec, for a 1.5 mL screw-cap tube), heated for 5 minutes, and then cooled to room temperature. The above thermally treated liquid (25 μL) and a PCR reaction solution (MMX, 75 μL) of AMPLICORE HBM (manufactured by Roche Diagnostics) were mixed in a 200 μL PCR tube, then mixed by light tapping the tube, and a nucleic acid amplification reaction was carried out using a thermal cycler (9600R, manufactured by Roche Diagnostics) The thermal cycler was operated under the following conditions.

Hold . . . at 50° C. for 2 minutes

Hold . . . at 96° C. for 5 minutes

Cycles 1 to 30 . . . at 96° for 20 seconds, at 58° C. for 20 seconds, and at 72° C. for 30 seconds

Hold . . . at 72° C. for 10 minutes

Hold . . . held at 72° C.

The resultant nucleic acid-amplified product was used according to the manual together with an avidin plate and various hybridizing reagents attached to AMPLICORE HBM. In this case, Columbus 2 (manufactured by Roche Diagnostics) was used as a microplate washer. Detection was conducted using an AMPLICORE HBM-dedicated plate reader (NJ-2300, manufactured by Roche Diagnostics).

Linearity Test

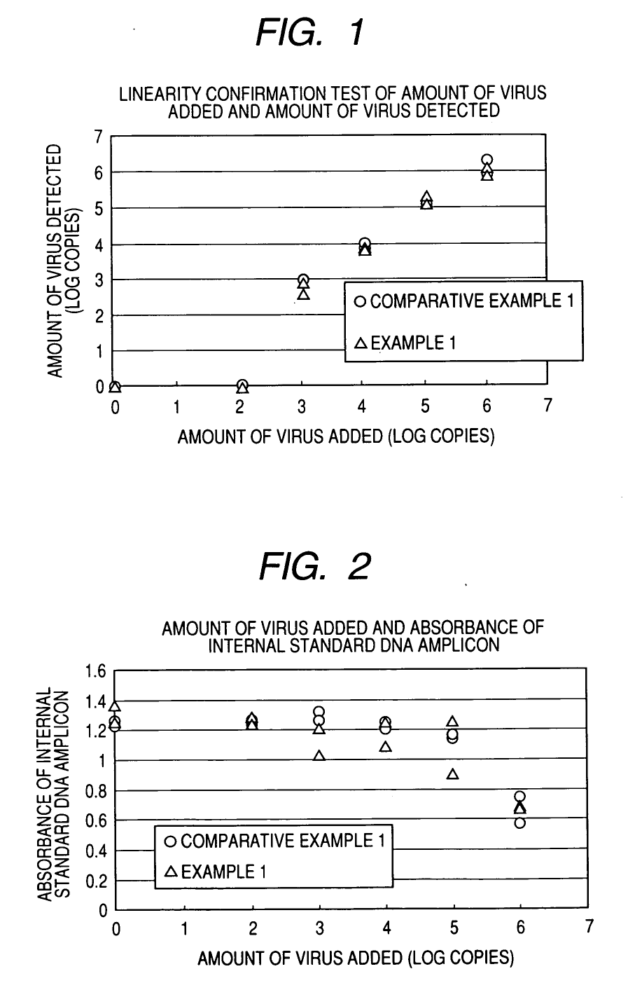

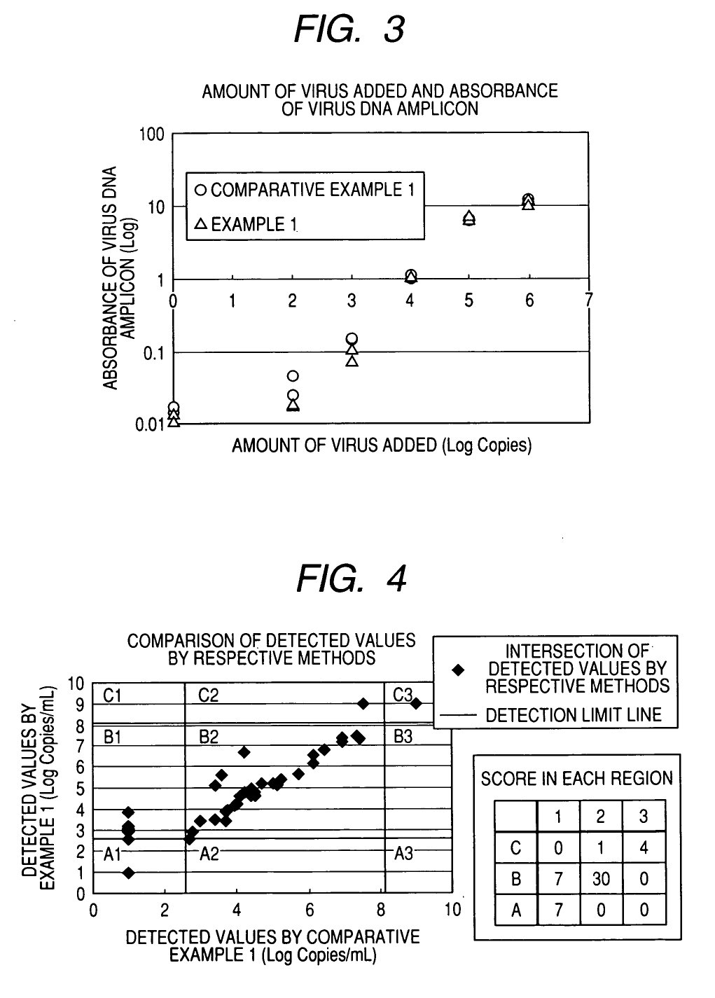

The analyte having each virus amount was measured by methods of Example 1 and Comparative Example 1 with N=2 in each case. The results are shown in Table 1, Table 2, and FIG. 1 to FIG. 3.

TABLE 1

| | Comparative Example 1 | | | | | |
|---|---|---|---|---|---|---|
| Amount of | Amount of virus | | | | | |
| virus added | detected | | Absorbance of | | Absorbance of | |
| (Log copies) | (Log copies) | | virus DNA | | internal standard | |
| 0 | 0 | 0 | 0.014 | 0.017 | 1.259 | 1.229 |
| 2 | 0 | 0 | 0.047 | 0.025 | 1.235 | 1.266 |
| 3 | 2.9 | 3 | 0.144 | 0.152 | 1.322 | 1.258 |
| 4 | 4 | 3.9 | 1.152 | 0.999 | 1.201 | 1.255 |
| 5 | 5.1 | 5.1 | 6.63 | 6.465 | 1.149 | 1.167 |
| 6 | 6.3 | 5.9 | 12.488 | 11.91 | 0.569 | 0.746 |

TABLE 2

| | Example 1 | | | | | |
|---|---|---|---|---|---|---|
| Amount of | Amount of virus | | | | | |
| virus added | detected | | Absorbance of | | Absorbance of | |
| (Log copies) | (Log copies) | | virus DNA | | internal standard | |
| 0 | 0 | 0 | 0.014 | 0.011 | 1.367 | 1.254 |
| 2 | 0 | 0 | 0.018 | 0.019 | 1.281 | 1.235 |
| 3 | 2.9 | 2.6 | 0.111 | 0.077 | 1.031 | 1.211 |
| 4 | 4 | 3.9 | 1.076 | 1.114 | 1.088 | 1.253 |
| 5 | 5.3 | 5.1 | 6.742 | 7.418 | 0.902 | 1.255 |
| 6 | 6.1 | 5.9 | 12.278 | 10.545 | 0.69 | 0.667 |

### Detection by Example 1 and Comparative Example 1 using Various Interference Substances-Added Human Normal Serum

An interference substance-added human normal serum was processed by the methods of Example 1 and Comparative Example 1.

A human normal serum to which each interference substance of bilirubin F, bilirubin C, hemolytic hemoglobin, and chyle was processed by both methods of Example 1 and Comparative Example 1. In both methods, detection sensitivity was not lowered by the addition of the interference substances and equal detection results were obtained.

### Detection Results by Each Method of Example 1 and Comparative Example 1 Using Clinical Specimen

49 clinical specimens were processed by each method of Example 1 and Comparative Example 1. The results are shown in Table 3 and FIG. 4.

With regard to the detection results, 1 means a value less than detection limit of the kit and 9 means a value more than detection limit of the kit.

In regions relatively correlative between both methods of Comparative Example 1 and Example 1, the specimens detected in a region (B2) within detection limit in both methods amounted to 30 specimens, the specimens detected in a region (C3) more than detection limit in both methods amounted to 4 specimens, and the specimens detected in a region (A1) less than detection limit in both methods amounted to 7 specimens. Moreover, the specimens judged to be particularly not correlative between the methods of Comparative Example 1 and Example 1 amounted to 4 specimens, which were detected in a region (C2) which was within detection limit in the method of Comparative Example 1 and was more than detection limit in the method of Example 1 and amounted to 7 specimens, which were detected in a region (B1) which was less than detection limit in the method of Comparative Example 1 and was within detection limit in the method of Example 1.

Of these, the 7 specimens which were detected in a region (B1) which was less than detection limit in the method of Comparative Example 1 and was within detection limit in the method of Example 1 were judged as negative in the method of Comparative Example 1 and as positive in the method of Example 1, and the results indicated that a pseudo negative detection result was obtained by the method of Comparative Example 1.

As above, it is indicated that the method of Example 1 can extract DNA of hepatitis B virus in high efficiency.

TABLE 3

| | | Method in |
|---|---|---|
| | Method in | Comparative |
| | Example 1 | Example 1 |
| | (Log copies) | (Log copies) |
| Clinical specimen 1 | 1(less than | 1(less than |
| | detection limit) | detection limit) |
| Clinical specimen 2 | 9(more than | 9(more than |
| | detection limit) | detection limit) |
| Clinical specimen 3 | 6.6 | 6.1 |
| Clinical specimen 4 | 2.6 | 1(less than |
| | | detection Unit) |
| Clinical specimen 5 | 9(more than | 9(more than |
| | detection limit) | detection limit) |
| Clinical specimen 6 | 5.1 | 3.4 |
| Clinical specimen 7 | 3.9 | 1(less than |
| | | detection limit) |
| clinical specimen 8 | 4.6 | 4.1 |
| Clinical specimen 9 | 4.9 | 4.4 |
| Clinical specimen 10 | 3.9 | 1(less than |
| | | detection limit) |
| Clinical specimen 11 | 7.3 | 7.4 |
| Clinical specimen 12 | 3.9 | 3.7 |
| Clinical specimen 13 | 1(less than | 1(less than |
| | detection limit) | detection limit) |
| Clinical specimen 14 | 1(less than | 1(less than |
| | detection limit) | detection limit) |
| Clinical specimen 15 | 1(less than | 1(less than |
| | detection limit) | detection limit) |
| Clinical specimen 16 | 5.1 | 5.1 |
| Clinical specimen 17 | 3.5 | 3.4 |
| Clinical specimen 18 | 1(less than | 1(less than |
| | detection limit) | detection limit) |
| Clinical specimen 19 | 2.9 | 1(less than |
| | | detection limit) |
| Clinical specimen 20 | 3.2 | 1(less than |
| | | detection limit) |
| Clinical specimen 21 | 3.1 | 1(less than |
| | | detection limit) |
| Clinical specimen 22 | 3.4 | 3.7 |
| Clinical specimen 23 | 3 | 1(less than |
| | | detection limit) |
| Clinical specimen 24 | 4.1 | 3.9 |
| Clinical specimen 25 | 6.8 | 6.4 |
| Clinical specimen 26 | 4.6 | 4.4 |
| Clinical specimen 27 | 1(less than | 1(less than |
| | detection limit) | detection limit) |
| Clinical specimen 28 | 7.4 | 6.9 |
| Clinical specimen 29 | 4.8 | 4.2 |
| Clinical specimen 30 | 4.2 | 4 |
| Clinical specimen 31 | 6.7 | 4.2 |
| Clinical specimen 32 | 2.6 | 2.7 |
| Clinical specimen 33 | 5.2 | 5 |
| Clinical specimen 34 | 1(less than | 1(less than |
| | detection limit) | detection limit) |
| Clinical specimen 35 | 4.6 | 4.5 |
| Clinical specimen 36 | 4.8 | 4.5 |
| Clinical specimen 37 | 5.2 | 4.7 |
| Clinical specimen 38 | 5.4 | 5.2 |
| Clinical specimen 39 | 9(more than | 9(more than |
| | detection limit) | detection limit) |
| Clinical specimen 40 | 9(more than | 7.5 |
| | detection limit) | |
| Clinical specimen 41 | 2.9 | 2.8 |
| Clinical specimen 42 | 5.7 | 5.7 |
| Clinical specimen 43 | 6.2 | 6.1 |
| Clinical specimen 44 | 3.5 | 3 |
| Clinical specimen 45 | 7.2 | 6.9 |
| Clinical specimen 46 | 1(less than | 1(less than |
| | detection limit) | detection limit) |
| Clinical specimen 47 | 5.6 | 3.6 |
| Clinical specimen 48 | 5 | 4.4 |
| Clinical specimen 49 | 7.5 | 7.3 |
| — | — | — |

Preparation of Magnet Unit for Automatic Nucleic Acid-Extracting Machine

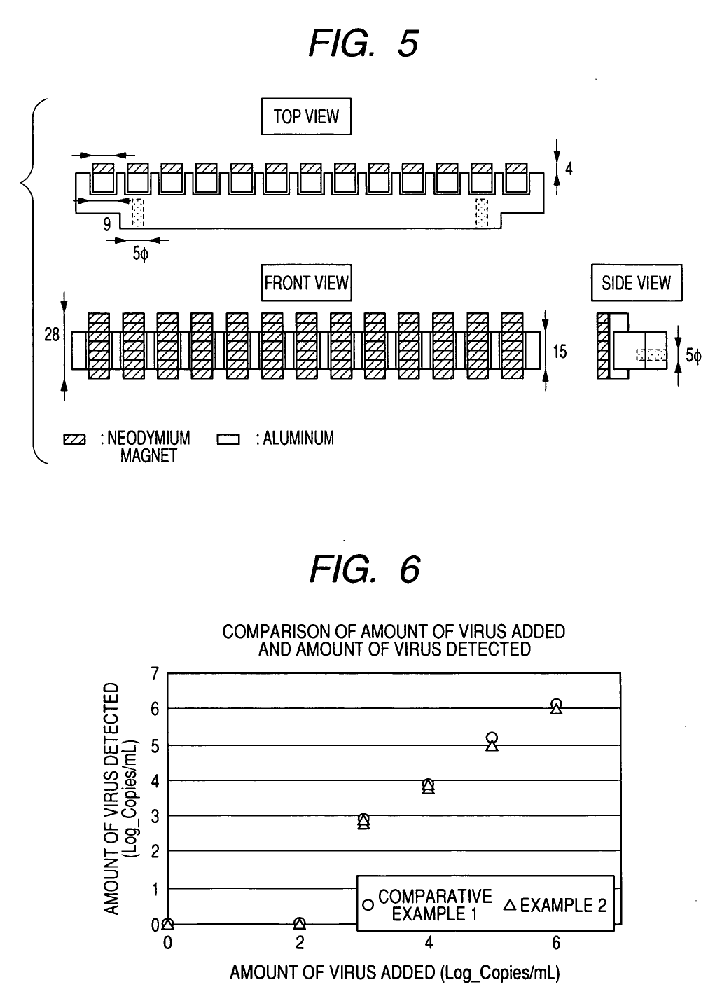

A magnet unit shown in FIG. 5, which can replace the magnet unit of the automatic nucleic acid-extracting apparatus MP12 (manufactured by Precision System Science) was prepared and mounted on MP12. As a magnet for a minimum constitutional unit, a square magnet having a size of 4 mm×4 mm×8 mm (manufactured by Niroku Seisakusho Co., Ltd.) was used.

### Example 2

Collection of Hepatitis B Virus by Polyethyleneimine-Immobilized Dextran-Coated Magnetic Fine Particles

1) The magnet unit of an automatic nucleic acid-extracting apparatus MP12 (manufactured by Precision System Science) was replaced by a magnet unit where 13 pieces of a magnet of 28 mm×4 mm×8 mm obtained by stacking 7 pieces of a square neodymium magnet of 4 mm×4 mm×8 mm were mounted in series.
2) The polyethyleneimine-immobilized dextran-coated magnetic fine particles (0.75% by weight, 10 μL) were placed in the second lane of a reaction tray of MP12.
3) A 0.25% physiological saline solution (20 μL) of polyacrylic acid is placed in the third lane of a reaction tray of MP12.
4) An aqueous polyethylene glycol solution for aggregation (40 μL) is placed in the fourth lane of a reaction tray of MP12.
5) An aqueous polyethylene glycol solution for washing (150 μL) is placed in the fifth lane of a reaction tray of MP12.
6) Ampdirect (trade name, manufactured by Shimadzu Corporation) is placed in the sixth lane of a reaction tray of MP12.
7) The reaction trays containing liquid of 2) to 6), a 1.5 mL screw-cap tube containing a plasma or serum of a subject which was expected to contain hepatitis B virus, and a tip for Binding/Free separation were provided on MP12.
8) A plasma or serial (50 μL) of a subject to be tested who was expected to be infected with hepatitis B virus was sucked and mixed with the polyethyleneimine-immobilized dextran-coated magnetic fine particles in the second lane of the tray, followed by pipetting for 60 seconds and any liquid was removed from the pipette.
9) The aqueous polyacrylic acid solution present in the third lane of the tray was sucked and added to the liquid in the second lane of the tray, followed by pipetting for 2 minutes.
10) The aqueous polyethylene glycol solution for aggregation present in the fourth lane of the tray was sucked and added to the liquid in the second lane of the tray, followed by pipetting for 60 seconds.
11) After 150 mL of the liquid in the tray 2 was sucked and the tip was migrated to a Binding/Free separation position, the magnet unit was migrated to the Binding/Free separation position and magnetic separation was conducted for 5 minutes in the tip to form pellets
12) A liquid separated from the pellets is discharged and removed from the tip and the magnet unit was returned to the original position.
13) The aqueous polyethylene glycol solution for washing present in the fifth lane of the tray was sucked and 50 μL of the air was sucked. After the magnet unit was migrated to the Binding/Free separation position, the solution was discharged and the magnet unit was returned to the original position.
14) Ampdirect (manufactured by Shimadzu Corporation) was sucked and pipetted to homogeneously disperse the pellets.
15) The resultant pellet dispersion was transferred into a heat block of MP12 set at 105° C. and heated for 8 minutes while 10 μL of pipetting was conducted.
16) The liquid subjected to the heat treatment was transferred into the first lane of the reaction tray.
17) The liquid was mixed with a nucleic acid-amplifying reagent of AMPLICORE HBM (manufactured by Roche Diagnostics) and a thermal cycler was set according to the method described in the procedure manual of AMPLICORE HBM (conditions the same as in Example 1) to amplify nucleic acids.
18) Detection (conditions the same as in Example 1) was conducted according to the method described in the procedure manual of AMPLICORE HBM.

### Detection Results by Each Method of Example 2 and Comparative Example 1

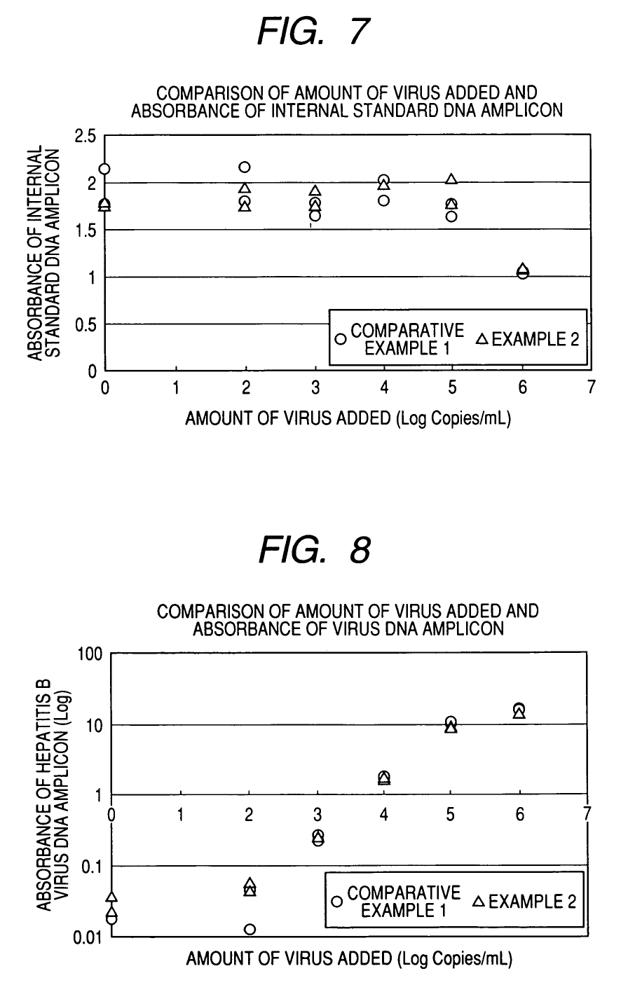

On the DNA extraction of hepatitis B virus in Example 2 using the automatic nucleic acid-extracting apparatus MP12 and the DNA extraction method by centrifugation in Comparative Example 1, a linearity test of the amount of virus detected and the amount thereof added was conducted. The results are shown in FIG. 6 to FIG. 8. It was confirmed that detection sensitivity equal to that in Comparative Example 1 was obtained also by the automated method in Example 2.

This application is based on Japanese patent application JP 2005-306008, filed on Oct. 20, 2005, the entire content of which is hereby incorporated by reference, the same as if set forth at length.

1. A water-soluble cationic magnetic fine particle comprising a substance having a cationic functional group, a substance having a hydroxyl group and a substance having magnetism, wherein the substance having a cationic functional group, the substance having a hydroxyl group and the substance having magnetism form a composite through a covalent bond or physical adsorption.

2. The water-soluble cationic magnetic fine particle according to claim 1, wherein the substance having magnetism is at least one substance selected from the group consisting of metals, metal oxides and latex magnetic beads, the substance having a hydroxyl group is a substance having a polyol framework, and the substance having a cationic functional group is at least one functional group selected from the group consisting of primary amino groups, secondary amino groups, tertiary amino groups, quaternary ammonium groups and guanidino groups.

3. The water-soluble cationic magnetic fine particle according to claim 1, wherein the substance having magnetism is at least one substance selected from the group consisting of magnetite, maghemite, hematite, gesite and latex magnetic beads,
the substance having a hydroxyl group is at least one polyol selected from the group consisting of dextran, dextrin, cellulose, agarose, starch, carboxymethyl cellolose, hydroxyacetyl cellulose, diethylaminoethyl cellulose, pullulan, amylose, gellan, arabinose galactan, polyvinyl alcohol and polyallyl alcohol, or a polyol obtained by polymerizing at least one compound selected from the group consisting of vinyl alcohol, allyl alcohol, 2-hydroxyethyl(meth)acrylate, glycerol-mono(meth)acrylate, 4-hydroxybutyl acrylate, 3-hydroxybutyl acrylate, 3-hydroxypropyl acrylate, and 2-hydroxy-2-methylpropyl acrylate as a component of a polymerizable monomer composition, and
the substance having a cationic functional group is at least one substance selected from polyallylamine, polyvinylamine, polyethyleneimine, polylysine, polyguanidine, poly(N,N-dimethylaminoethyl(meth)acrylamide), poly(N,N-dimethylaminopropyl(meth)acrylamide), polyaminopropyl(meth)acrylamide, or a substance obtained by substituted with at least one compound selected from the group consisting of diethylaminoethyl chloride hydrochloride, ethylenediamine, hexamethylenediamine, tris(aminoethyl)amine, aziridine hydrochloride, aminopropyltriethoxysilane and aminoethylaminopropyltriethoxysilane.

4. The water-soluble cationic magnetic fine particle according to claim 1, wherein the substance having a cationic functional group is at least one substance selected from polyethyleneimine and polylysine, and the substance having a hydroxyl group is at least one substance selected from dextran and polyvinyl alcohol, and the substance having magnetism is at least one substance selected from magnetite and maghemite.

5. A combined body of a water-soluble cationic magnetic fine particle and a phospholipid vesicle, wherein the water-soluble cationic magnetic fine particle according to claim 1 and a body having a phospholipid membrane (phospholipid vesicle) are combined.

6. The combined body according to claim 5, wherein the phospholipid vesicle is a virus, a bacterium, a fungus, or a true fungus.

7. A combined body of a water-insoluble cationic magnetic fine particle, a phospholipid vesicle and a masking agent, wherein the combined body according to claim 5 and a masking agent are combined.

8. The combined body according to claim 7 wherein the masking agent is a substance containing at least one acid structure selected from the group consisting of carboxylic acid, phosphoric acid, sulfuric acid, and boric acid.

9. A composite of a water-insoluble cationic magnetic fine particle, a phospholipid vesicle and an aggregating agent, wherein the combined body according to claim 5 and an aggregating agent are combined.

10. A composite of a water-insoluble cationic magnetic fine particle, a phospholipid vesicle, a masking agent and an aggregating agent, wherein the combined body according to claim 7 and an aggregating agent are combined.

11. The composite according to claim 9, wherein the aggregating agent is a polyether.

12. The composite according to claim 9, wherein the aggregating agent is at least one substance selected from the group consisting of a substance having a polyalkylene glycol structure in a main chain, a substance having a polyalkylene glycol structure in a side chain, and a substance having a polyglycerin structure in a main chain.

13. The composite according to claim 12, which is a composite of a cationic magnetic fine particle, a phospholipid vesicle, a masking agent and an aggregating agent,
wherein the cationic magnetic fine particle is a composite of dextran-coated magnetite and polyethyleneimine,
the phospholipid vesicle is a virus,
the masking agent is at least one masking agent selected from the group consisting of poly(meth)acrylic acid, polycarboxymethylstyrene, hyaluronic acid, α-polyglutamic acid, ω-polyglutamic acid, gelan, carboxymethyl cellulose, carboxymethyl dextran, polyphosphoric acid, poly(phosphoric acid sugar), nucleic acids, phosphoric acid, citric acid, polystyrylsulfuric acid, dextran sulfuric acid and polystyrylboric acid, and
the aggregating agent is at least one aggregating agent selected from the group consisting of polyethylene glycol, polypropylene glycol, polyethyleneglycol-polypropylene glycol random copolymer, and polyethyleneglycol-polypropylene glycol block copolymer, polymethoxyethoxy(meth)acrylate, poly(diethylene glycol-(meth)acrylate-methyl ether), poly(triethylene glycol-(meth)acrylate-methyl ether), poly(tetraethylene glycol-(meth)acrylate-methyl ether), poly(polyethylene glycol(meth)acrylate), and random and block copolymers thereof, and poly(glycerin-2-ethyl ether), poly(glycerin-2-diethylene glycol methyl ether), poly(glycerin-2-triethylene glycol methyl ether), poly(glycerin-2-tetraethylene glycol methyl ether), poly(glycerin-2-polyethylene glycol ether), poly(glycerin-2-polypropylene glycol ether), and poly(glycerin-2-polyethylene glycol ether)(glycerin-2-polypropylene glycol ether) copolymer.

14. The composite according to claim 12, which is a composite of a cationic magnetic fine particle, a phospholipid vesicle, a masking agent and an aggregating agent,
wherein the cationic magnetic fine particle is a composite of magnetite coated with dextran having an average molecular weight of 3,000 to 100,000 and polyethyleneimine having an average molecular weight of 600 to 10,000,
the phospholipid vesicle is at least one virus selected from the group consisting of influenza virus, cytemegalo virus, HIV, papilloma virus, respiratory syncytial virus, poliomyelitis virus, pox virus, measles virus, arbovirus, coxsackievirus, herpes virus, hantavirus, hepatitis virus, Lyme disease virus, mumps virus and rotavirus,
the masking agent is poly(meth)acrylic acid having an average molecular weight of 1 0,000 to 50,000 or a salt thereof, and
the aggregating agent is polyethylene glycol having an average molecular weight of 2,000 to 20,000.

15. A process for separating a phospholipid vesicle, comprising mixing an aqueous solution of a water-soluble cationic magnetic fine particle containing a substance having a cationic functional group, a substance having a hydroxyl group and a substance having magnetism, with a liquid containing a phospholipid vesicle, to form a water-soluble combined body of a cationic magnetic fine particle and a phospholipid vesicle.

16. The process for separating a phospholipid vesicle according to claim 15, which further comprises mixing with a masking agent.

17. The process for separating a phospholipid vesicle according to claim 15, which comprises:
an adsorption step of mixing a water-soluble cationic magnetic fine particle having a polyol and a substance having a cationic functional group in the structure, with a liquid containing a phospholipid vesicle to form a water-soluble combined body of a cationic magnetic fine particle and a phospholipid vesicle;
an aggregation step of mixing the water-soluble combined body with an aggregating agent to form a water-insoluble composite of a cationic magnetic fine particle, a phospholipid vesicle and an aggregating agent;
a separation step of forming a pellet of the water-insoluble composite by at least one method selected from magnetic separation, centrifugation and filtration and removing the resultant supernatant; and
a re-dispersion step of dispersing the pellet in a liquid.

18. The process for separating a phospholipid vesicle according to claim 15, which comprises:
an adsorption step of mixing a water-soluble cationic magnetic fine particle having a polyol and a substance having a cationic functional group in the structure, with a liquid containing a phospholipid vesicle to form a water-soluble combined body of a cationic magnetic fine particle and a phospholipid vesicle;
a masking step of mixing the water-soluble combined body with an aqueous solution containing a masking agent to form a water-soluble combined body of a cationic magnetic fine particle, a phospholipid vesicle and a masking agent;
an aggregation step of mixing the water-soluble combined body of a cationic magnetic fine particle, a phospholipid vesicle and a masking agent, with an aggregating agent to form a water-insoluble composite of a cationic magnetic fine particle, a phospholipid vesicle, a masking agent and an aggregating agent;
a separation step of forming a pellet of the water-insoluble composite by at least one method selected from magnetic separation, centrifugation and filtration, and removing the resultant supernatant; and
a re-dispersion step of dispersing the pellet in a liquid.

19. A process for detecting a virus, comprising a step of mixing a water-soluble cationic magnetic fine particle containing a substance having a cationic functional group, a substance having a hydroxyl group and a substance having magnetism, with a liquid containing a virus to form a water-soluble combined body of a cationic magnetic fine particle and a phospholipid vesicle.

20. The process for detecting a virus according to claim 19, which comprises:
a step of mixing a water-soluble cationic magnetic particle, with a serum or plasma containing the virus to form a water-soluble combined body of a cationic magnetic fine particle and virus, in which the water-soluble cationic magnetic particle is obtained by treating a water-soluble dextran magnetite with a periodate to form a dextran magnetite having an aldehyde and then covalently bonding the dextran magnetite having an aldehyde through reductive amination with polyethyleneimine having an average molecular weight of 1,800 which is a substance having a cationic functional group;
a step of mixing the water-soluble combined body with an aqueous solution of polyacrylic acid having an average molecular weight of 25,000 to form a water-soluble combined body of a cationic magnetic fine particle, virus and polyacrylic acid;
a step of further mixing the water-soluble combined body of a cationic magnetic fine particle, virus and polyacrylic acid, with an aqueous solution of polyethylene glycol having a molecular weight of 6,000 to 8,000 to form a water-insoluble composite of a cationic magnetic fine particle, virus, polyacrylic acid and polyethylene glycol;
a step of forming a pellet of the water-insoluble composite by magnetic collection and removing the resultant supernatant;
a step of dispersing the pellet in a nucleic acid amplification reaction solution;
a step of denaturing the virus in the pellet by heating to release nucleic acids of the virus; and
a step of amplifying the virus nucleic acids by a nucleic acid amplification reaction (PCR, ICAN).
